(19) United States
(12) Patent Application Publication
(10) Pub. No.: US 2025/0113849 A1
(43) Pub. Date: Apr. 10, 2025

(54) COMPOSITIONS AND METHODS FOR ACTIVATING PHYSIOLOGICAL HEALTH DEFENSES

(71) Applicant: QUINTESSENTIALI, LLC, Cambridge, MA (US)

(72) Inventor: William W. LI, Natick, MA (US)

(73) Assignee: QUINTESSENTIALI, LLC, Cambridge, MA (US)

(21) Appl. No.: 18/726,304

(22) PCT Filed: Jan. 3, 2023
(86) PCT No.: PCT/US2023/060027

§ 371 Date: Jul. 2, 2024

Related U.S. Application Data
(60) Provisional application No. 63/296,060, filed on Jan. 3, 2022.

(51) Int. Cl. A23L 27/00; A23L 2/38

(52) U.S. Cl. CPC A23L 27/00; A23L 2/38

(57) ABSTRACT

The present invention provides compositions and methods for activating physiological health defenses. The invention, comprising combinations of, and methods of providing and administering, (1) anthocyanins; (2) β-(1-3,1-6)-D-glucan; (3) chlorogenic acid; (4) curcumin; (5) epigallocatechin-3-gallate (EGCG); (6) genistein; (7) hydroxytyrosol; (8) lycopene (including both cis- and trans-isomers); (9) Lactobacillus reuteri, Akkermansia mucinophila, and/or Ruminoccocus sp. bacteria; (10) omega-3 polyunsaturated fatty acid (PUFA); (11) quercetin; and (12) proanthocyanidins, relieves and alleviates numerous deleterious health conditions and/or to support mammalian physiological health defenses including (but not limited to) angiogenesis, regeneration, microbiome, DNA protection, immunity, metabolism, and adaptive thermogenesis.

### CROSS-REFERENCE TO RELATED APPLICATION

This application claims the benefit of a U.S. provisional application filed on Jan. 3, 2022 in the United States Patent and Trademark Office, which was assigned Ser. No. 63/296,060, which is hereby incorporated by reference in its entirety.

### FIELD OF THE INVENTION

The present invention relates generally to the field of dietary supplements, food additives, and therapeutic compositions and methods.

### BACKGROUND OF THE INVENTION

The present invention provides compositions and methods for activating physiological health defenses. The invention is designed to relieve and alleviate numerous deleterious health conditions and/or to support mammalian physiological health defenses including (but not limited to) angiogenesis, regeneration, microbiome, DNA protection, and immunity, metabolism, and adaptive thermogenesis.

Administering some naturally occurring compositions can have positive physiological effects. However, administering such compositions in isolation may not achieve optimal results. It may be preferable to administer such compositions in an admixture. Such an admixture may improve over the isolated compositions each component's solubility, stability, bioavailability, and potency. The combination may have co-pharmacological interactions achieving enhanced physiological effects, and also may improve the flavor profile over the isolated compositions. Further, the preferred admixture may depend on specific context and needs, for example a food product intended to be cooked will require the composition to have high chemical stability, or an individual component's solubility and absorption profile may improve when combined with lipophilic elements in a composition.

The present invention achieves optimal combinatorial effects to enhance and optimize desired characteristics.

### SUMMARY OF THE INVENTION

The present invention provides compositions and methods for activating mammalian physiological health defenses, such as angiogenesis, regeneration, microbiome, DNA protection, immunity, metabolism, and adaptive thermogenesis.

The present invention generally comprises methods and compositions comprising a selection from twelve entities: (1) anthocyanins; (2) β-(1-3,1-6)-D-glucan; (3) chlorogenic acid; (4) curcumin; (5) epigallocatechin-3-gallate (EGCG); (6) genistein; (7) hydroxytyrosol; (8) lycopene (including both cis- and trans-isomers); (9) Lactobacillus reuteri, Akkermansia mucinophila, and/or Ruminoccocus sp. bacteria; (10) omega-3 polyunsaturated fatty acid (PUFA); (11) quercetin; and (12) proanthocyanidins. In preferred embodiments, the invention comprises compositions comprising a selection of three or more of the twelve entities, in isolated form or as present in their native food source. As elaborated in the paragraphs below, the invention further includes a method of providing compositions comprising a selection from the above twelve entities, and a method of activating physiological health defenses by administering a product comprised of a selection of three or more of the above twelve entities.

Each of these twelve entities has documented physiological benefits ranging from, e.g., antioxidant effects, improving gastrointestinal microbiome with subsequent physiological and mental health benefits, lowering serum lipid levels, improving glucose metabolism, improving endothelial function, improving neovasculation, improving granulation and healing, reducing adipose mass, improving adipose composition, activating brown adipose cells for thermogenesis and increasing metabolism, inhibiting angiogenesis to prevent cancers and vision loss, mobilizing progenitor cells for tissue repair and regeneration, fighting microbial and viral infections, and ameliorating inflammation. However, by selecting a combination of these entities for specific applications, it is possible to enhance and optimize these benefits by multi-targeting of the body's physiological health defenses, specifically, angiogenesis, stem cells, microbiome, DNA protection, immune function, metabolism, and adaptive thermogenesis, while also optimizing other desired characteristics such as, e.g., solubility, the desired flavor profile, volume constraints, and heating and cooking requirements, and other desirable attributes. For example, combining lycopene with omega-3 polyunsaturated fatty acid (PUFA) and chlorogenic acid improves systemic absorption of lycopene to provide antiangiogenic, anti-inflammatory, protecting neural progenitor cells, while also reducing the differentiation of white adipocyte progenitor cells, and improving systemic production of nitric oxide which lowers blood pressure while improving vascular endothelial cell health.

Each of the twelve entities possesses well-defined molecular, biochemical, and cellular mechanisms of action, and evidence for biological effect from in vitro, in vivo models and/or human clinical testing assessing activity for angiogenesis, regeneration, microbiome, DNA protection, immunity, and adaptive thermogenesis.

The invention implements deep functional knowledge of the twelve entities, and surprising and unexpected chemistry and pharmacokinetics of each entity, and the foods in which they are present; biological interactions between each entity and the five physiological defense systems (i.e., angiogenesis, regeneration, microbiome, DNA protection, and immunity); operational understanding of the physiological defense systems and how they relate to health and disease; data analytical techniques for assessing the physiological defense systems; combinatorial properties of the entities; and end characteristics of the combinations, such as flavor, solubility, stability, potency, and bioavailability, among others. For example, isolated quercetin is known to be bitter in flavor. Quercetin in combination with other of the twelve entities, however, may unexpectedly produce a palatable, desirable flavor profile. The five physiological health defenses (i.e., angiogenesis, regeneration, microbiome, DNA protection, and immunity) are further described in WILLIAM W. LI, MD, EAT TO BEAT DISEASE: THE NEW SCIENCE OF HOW YOUR BODY CAN HEAL ITSELF (1st ed. 2019), which is incorporated by reference herein in its entirety.

The composition and method of the present invention may be employed to create dietary supplements intended to support and activate mammalian physiological defense systems; to create novel food products that have desirable flavor profiles and support and activate physiological defense systems, such as flavorings, spice packs, oil packs, meal replacements, functional beverages, functional foods, medical foods, condiments, prescription diets, pet foods; and to create novel pharmaceutical products intended to prevent, treat, or modify diseases in mammals, including human patients. The composition and method of the present invention may also be ideal to create dietary sustenance, foods, and/or supplements for human and/or animal space travel, space station settlement, and extraterrestrial settlement, i.e., moon settlement or interplanetary settlement (e.g., Mars).

The invention may be used to (A) optimize body condition—support the body's intrinsic ability to fight adiposity and visceral fat, enhance browning of fat, oppose weight gain, foster weight loss, fight obesity, improve metabolism; (B) optimize vascular health—support the body's intrinsic ability to maintain endothelial health and function; support endothelial repair and regeneration, stimulate physiological angiogenesis, inhibit pathological angiogenesis; (C) improve brain function—support and improve the body's intrinsic ability to maintain cognition, increase alertness, oppose dysphoria, promote euphoria; (D) improve gut health—support and improve the gut microbiome to healthy organisms and ecosystem; (E) slow and prevent aging—support and improve the body's intrinsic ability to control and slow cellular aging, reduce oxidative stress; (F) boost immunity—support and improve the body's intrinsic ability to optimize innate and adaptive immunity; and (G) reduce inflammation—support and improve the body's intrinsic ability to reduce the intensity of inflammation to protect against cell and organ injury while defending against pathology.

The invention comprises compositions composed of a combination of a selection from twelve entities: (1) anthocyanins; (2) β-(1-3,1-6)-D-glucan; (3) chlorogenic acid; (4) curcumin; (5) epigallocatechin-3-gallate (EGCG); (6) genistein; (7) hydroxytyrosol; (8) lycopene (including both cis- and trans-isomers); (9) Lactobacillus reuteri, Akkermansia mucinophila, and/or Ruminoccocus sp. bacteria; (10) omega-3 polyunsaturated fatty acid (PUFA); (11) quercetin; and (12) proanthocyanidins. In preferred embodiments, the combination comprises three or more of the above-listed entities. In some embodiments, the combination may comprise four or more, five or more, six or more, seven or more, eight or more, nine or more, ten or more, eleven, or all twelve of the above-listed entities.

The invention further comprises a method of providing a composition for the activation of physiological health defenses in a mammal, comprising selecting a combination of the twelve entities (1) anthocyanins; (2) β-(1-3,1-6)-D-glucan; (3) chlorogenic acid; (4) curcumin; (5) epigallocatechin-3-gallate (EGCG); (6) genistein; (7) hydroxytyrosol; (8) lycopene (including both cis- and trans-isomers); (9) Lactobacillus reuteri, Akkermansia mucinophila, and/or Ruminoccocus sp. bacteria; (10) omega-3 polyunsaturated fatty acid (PUFA); (11) quercetin; and (12) proanthocyanidins. In preferred embodiments, the method comprises selected three or more of the above-listed entities. In some embodiments, the method may comprise selecting four or more, five or more, six or more, seven or more, eight or more, nine or more, ten or more, eleven, or all twelve of the above-listed entities.

The invention further comprises a method of activating physiological defenses in a mammalian subject comprising the steps of: (I) selecting a composition for the activation of physiological defenses in a mammal, using a combination of the twelve entities (1) anthocyanins; (2) β-(1-3,1-6)-D-glucan; (3) chlorogenic acid; (4) curcumin; (5) epigallocatechin-3-gallate (EGCG); (6) genistein; (7) hydroxytyrosol; (8) lycopene (including both cis- and trans-isomers); (9) Lactobacillus reuteri, Akkermansia mucinophila, and/or Ruminoccocus sp. bacteria; (10) omega-3 polyunsaturated fatty acid (PUFA); (11) quercetin; and (12) proanthocyanidins; and (II) administering the selected combination of three or more of the above-listed twelve entities to a mammalian subject. In some embodiments, step (I) of the method may comprise selecting four or more, five or more, six or more, seven or more, eight or more, nine or more, ten or more, eleven, or all twelve of the above-listed entities.

The invention further comprises a food product, wherein the food product may be a human food product, a dietary supplement formulated for humans and/or non-human mammals, or a pet food. The food product may comprise at least three of the twelve functional entities selected from (1) anthocyanins; (2) β-(1-3,1-6)-D-glucan; (3) chlorogenic acid; (4) curcumin; (5) epigallocatechin-3-gallate (EGCG); (6) genistein; (7) hydroxytyrosol; (8) lycopene (including both cis- and trans-isomers); (9) Lactobacillus reuteri, Akkermansia mucinophila, and/or Ruminoccocus sp. bacteria; (10) omega-3 polyunsaturated fatty acid (PUFA); (11) quercetin; and (12) proanthocyanidins. The food product may be a functional food. The food product may comprise a soup, which may be dehydrated or hydrated, and may comprise broth, or may comprise at least broth and noodles. The twelve functional entities may be incorporated into the soup as extracts, synthetics, or in a whole food selected for its high concentration of at least one of the twelve functional entities (e.g., tomatoes having a high concentration in lycopene).

In still another aspect, the invention further comprises a noodle soup kit, wherein the kit comprises dehydrated noodles, a dehydrated soup base, and at least three of functional entities, wherein the functional entities are selected from the group consisting of: anthocyanins; β-(1-3,1-6)-D-glucan; chlorogenic acid; curcumin; epigallocatechin-3-gallate (EGCG); genistein; hydroxytyrosol; lycopene (including both cis- and trans-isomers); Lactobacillus reuteri, Akkermansia mucinophila, and/or Ruminoccocus sp. bacteria; omega-3 polyunsaturated fatty acid (PUFA); quercetin; and proanthocyanidins. In the noodle soup kit, the twelve functional entities may be incorporated into the soup as extracts, synthetics, or in a whole food selected for its high concentration of at least one of the twelve functional entities (e.g., tomatoes having a high concentration in lycopene).

### BRIEF DESCRIPTION OF THE DRAWINGS

FIGS. 1A-1C depict a schema of the present invention and its uses.

More particularly, FIG. 1A depicts the array of twelve compositions for activating physiological defenses, of which three or more are selected and combined for a particular purpose. FIG. 1B depicts the health systems, of which one or more is activated according to the invention of the present disclosure. FIG. 1C depicts two physiological results from the health defense system activation, including health maintenance and improvement, and suppression, treatment, and/or reversal of disease state.

FIG. 2 depicts a flow diagram for a method of designing natural product platforms for foods and supplements that deliver naturally occurring bioactives with health supporting properties.

FIG. 3 depicts a categorized non-limiting list of sources for generating natural product platforms for foods and supplements that deliver naturally occurring bioactives with health supporting properties.

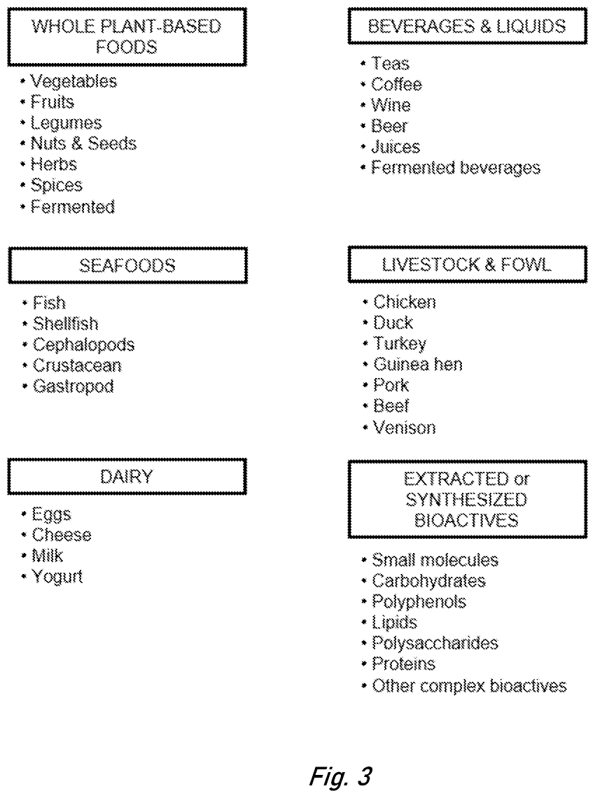

### DETAILED DESCRIPTION OF THE INVENTION

As used herein, including the appended claims, the singular forms of words such as “a,” “an,” and “the,” include their corresponding plural references unless the context clearly dictates otherwise.

All references cited herein are incorporated by reference to the same extent as if each individual publication, patent application, or patent, was specifically and individually indicated to be incorporated by reference.

The present invention generally comprises methods and compositions comprising a selection from twelve entities: (1) anthocyanins; (2) β-(1-3,1-6)-D-glucan; (3) chlorogenic acid; (4) curcumin; (5) epigallocatechin-3-gallate (EGCG); (6) genistein; (7) hydroxytyrosol; (8) lycopene (including both cis- and trans-isomers); (9) Lactobacillus reuteri, Akkermansia mucinophila, and/or Ruminoccocus sp. bacteria; (10) omega-3 polyunsaturated fatty acid (PUFA); (11) quercetin; and (12) proanthocyanidins. In preferred embodiments, the invention comprises compositions comprising a selection of three or more of the twelve entities. The invention further includes a method of providing compositions comprising a selection from the above twelve entities, and a method of activating physiological health defenses comprising administering combinations of the above twelve entities.

Each of the twelve entities possesses well-defined molecular, biochemical, and cellular mechanisms of action, and evidence in animal models and/or human preclinical and clinical testing for biological activity in angiogenesis, regeneration, microbiome, DNA protection, immunity, metabolism, and adaptive thermogenesis.

Anthocyanins are a genus of water-soluble flavonoid phytopigments. The genus encompasses compounds having a 2-phenyl-benzopyran backbone, such as that in formula (I) below:

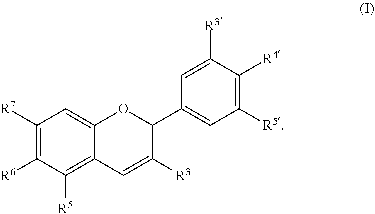

The substituent groups may comprise, independently, any of: hydrogen, —OCH₃, —OH, and any glycoside (including glucoside). The backbone may be neutrally charged or cationic, positively charged at the benzopyran oxygen.

Accordingly, anthocyanins may be subdivided into anthocyanin glycosides and anthocyanidin aglycones. Examples of anthocyanins include pelargonidin-3-glucoside, cyanidin-3-glucoside, delphinidin-3-glucoside, and rosinidin-3-glucoside.

Anthocyanins inhibit pathological angiogenesis; protect and stimulate progenitor cells; improve the gut microbiome; protect DNA from oxidative damage, induce beneficial epigenetic changes; suppress inflammation; enhance immunity; slow cellar aging by protecting telomere length; activate brown adipose tissue leading to improved metabolism; lowers visceral adiposity; target cancer stem cells.

Beta-(1-3,1-6)-D-glucan is a branching polysaccharide film that may be derived from fungi cell wall, including yeast cell wall. β-D-glucans are polysaccharides occurring in cell walls of bacteria, cereals, fungi, and other organisms. Glucans comprise six-carbon D-glucose monomers. The polymers can be characterized by which carbon atoms on the ring bond with the adjacent monomer. β-(1-3,1-6)-D-glucan is a branching polysaccharide comprising a mixture of 1-3 carbon-carbon bonded D-glucose monomers and 1-6 carbon-carbon bonded D-glucose monomers.

Beta-(1-3,1-6)-D-glucan inhibits pathological angiogenesis; stimulates physiological angiogenesis; improves gut microbiome composition; protects DNA against oxidative damage; slow cellular aging by protecting telomere length; enhances immune function; exerts anti-inflammatory effects; causes adipocyte browning; increases adaptive thermogenesis; and reduces visceral obesity.

Chlorogenic acid is an ester of caffeic acid and 3-hydroxy-L-quinic acid.

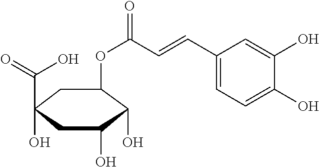

It is found in numerous plants, including, e.g., common bamboo and heather, peaches, plums, and coffee bean. Chlorogenic acid possesses significant physiological and medicinal benefits including: inhibition of pathological angiogenesis; stimulation of physiological angiogenesis; improves gut microbiome composition; protects DNA against oxidative damage; slow cellular aging by protecting telomere length; enhances immune function; exerts anti-inflammatory effects; causes adipocyte browning; increases adaptive thermogenesis; reduces visceral obesity; interferes with cellular binding sites of SARS-CoV-2 on human cells.

Curcumin is a diphenolic yellow phytopigment, usually extracted from Curcuma longa (turmeric plant).

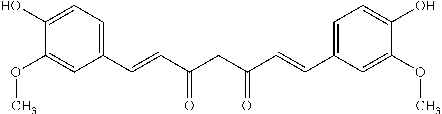 [SMILES: COc1cc(/C=C/C(=O)CC(=O)/C=C/c2ccc(O)c(OC)c2)ccc1O]

Curcumin possesses significant physiological and medicinal benefits including: inhibition of pathological angiogenesis; stimulation of physiological angiogenesis; improves gut microbiome composition; protects DNA against oxidative damage; slow cellular aging by protecting telomere length; enhances immune function; exerts anti-inflammatory effects; causes adipocyte browning; increases adaptive thermogenesis; and reduces visceral obesity and abdominal fat.

Epigallocatchin-3-gallate (EGCG) is an ester of the flavanol epigallocatechin and trihydroxybenzene compound gallic acid. It is found in high concentrations in tea.

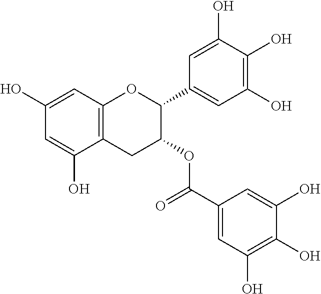 [SMILES: O=C(O[C@@H]1Cc2c(O)cc(O)cc2O[C@@H]1c1cc(O)c(O)c(O)c1)c1cc(O)c(O)c(O)c1]

Epigallocatchin-3-gallate possesses significant physiological and medicinal benefits including: inhibition of pathological angiogenesis; stimulation of physiological angiogenesis; improves endothelial health and vascular function; stimulates progenitor cells in regeneration; targets cancer stem cells; improves gut microbiome composition; protects DNA against oxidative damage; slow cellular aging by protecting telomere length; enhances immune function; exerts anti-inflammatory effects; causes adipocyte browning; increases adaptive thermogenesis; and reduces visceral obesity and abdominal fat.

Genistein (a/k/a prunetol) is an isoflavone phytochemical, known to have angiogenesis inhibitor and epidermal growth factor receptor (EGFR) inhibitor activities. Genistein has been found in Genista tinctoria, Trifolium sp., Flemingia sp., lupin, soybean, and psoralea, among others. Genistein possesses significant physiological and medicinal benefits including: inhibition of pathological angiogenesis; stimulation of physiological angiogenesis; improves endothelial health and vascular function; stimulates progenitor cells in regeneration; decreases cancer cell populations; has cancer chemopreventive effects; improves gut microbiome composition; protects DNA against oxidative damage; prevents telomere

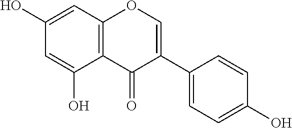 [SMILES: O=c1c(-c2ccc(O)cc2)coc2cc(O)cc(O)c12]

maintenance in cancer cells; enhances immune function; exerts anti-inflammatory effects; causes adipocyte browning; increases adaptive thermogenesis; and improves metabolism, and reduces adipocyte accumulation

Hydroxytyrosol (“HXT”) (4-(2-hydroxyethyl)-1,2-benzenediol) is a powerful antioxidant phenylethanoid known the be found in olive leaves, olive pulp, and olive oil.

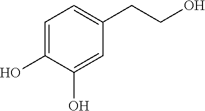 [SMILES: OCCc1ccc(O)c(O)c1]

HXT also has lipid reduction activity. However, HXT reacts with food substrates and is known to cause strong flavor and odor. Therefore, it would be greatly beneficial to develop improved HXT compositions and admixtures that do not cause strong flavor and odor. HXT possesses significant physiological and medicinal benefits including: inhibition of pathological angiogenesis; stimulation of physiological angiogenesis; protects endothelial health against inflammation; stimulates progenitor cells in regeneration; targets cancer stem cells; improves gut microbiome composition; protects DNA against oxidative damage; slow cellular aging by protecting telomere length; exerts anti-inflammatory effects; ameliorates metabolic dysfunction; increases adaptive thermogenesis; and suppresses diet-induced obesity. Furthermore, HXT has demonstrated effectiveness interfering with the receptor binding activity of SARS-CoV-2 spike protein. See, e.g., Mahmut Çerkez Ergören et al., A pilot study on the preventative potential of alpha-cyclodextrin and hydroxytyrosol against SARS-CoV-2 transmission, ACTA BIOMED. 2020; 91(Supp. 13); Yohei Takeda et al., The SARS-CoV-2-Inactivating Activity of Hydroxytyrosol-Rich Aqueous Olive Pulp Extract (Hidrox®) and Its Use as a Virucidal Cream for Topical Application, VIRUSES, 2021 13(2), 232.

Lycopene is a carotenoid hydrocarbon pigment generally found in red fruits and vegetables such as, e.g., carrot, watermelon, grapefruit, goji, papaya, and, especially, tomato.

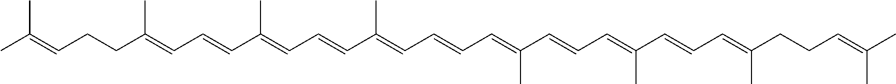 [SMILES: CC(C)=CCC/C(C)=C/C=C/C(C)=C/C=C/C(C)=C/C=C/C=C(C)/C=C/C=C(C)/C=C/C=C(\C)CCC=C(C)C]

Lycopene comes in numerous trans- and cis-isomers, depending on the positions of the C═C double bonds. All-trans-lycopene is a straight hydrocarbon. The various cis-isomers have a bent shape. Lycopene has positive cardiovascular health benefits, and cancer chemopreventative effects. Lycopene also possesses significant physiological and medicinal benefits including: inhibition of pathological angiogenesis; improves endothelial health and vascular function; stimulates progenitor cells in regeneration; decreases cancer cell populations; has cancer chemopreventive effects; improves gut microbiome composition; protects DNA against oxidative damage; assists with telomere length maintenance; enhances immune function; exerts anti-inflammatory effects; causes adipocyte browning; reduces weight gain; increases adaptive thermogenesis; and improves metabolism and reduces abdominal adipose accumulation.

Omega-3 polyunsaturated fatty acid (PUFA) are PUFAs characterized by having a C═C double bond three atoms away from the terminal methyl group on the fatty acid chain.

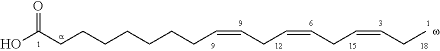 [SMILES: CC/C=C\C/C=C\C/C=C\CCCCCCCC(=O)O]

Omega-3 PUFAs occur in high concentrations in marine organisms such as fish as well as in certain plant-based foods such as flax seeds, tree nuts, chia seeds, and legumes. Consuming omega-3 PUFAs is associated with significant cardiovascular health benefits. Omega-3 PUFAs possesses significant physiological and medicinal benefits including: inhibition of pathological angiogenesis; stimulation of physiological angiogenesis; improves endothelial health and vascular function; stimulates progenitor cells in regeneration; decreases cancer cell populations; has cancer chemopreventive effects; improves gut microbiome composition; protects DNA against oxidative damage; attenutation of telomere shortening and cellular aging; enhances immune function; exerts anti-inflammatory effects; causes adipocyte browning; increases adaptive thermogenesis; improves metabolism; and prevents weight gain; promotes weight reduction.

Quercetin is a flavanol found in numerous fruits, vegetables, seeds, grains, and the like.

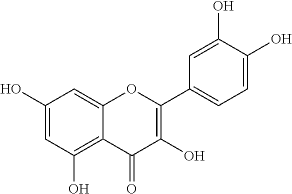 [SMILES: O=c1c(O)c(-c2ccc(O)c(O)c2)oc2cc(O)cc(O)c12]

Quercetin is a polar auxin transport inhibitor in plants, and has been associated with cancer prevention properties in mammals. Quercetin is bitter in flavor, and therefore it would be beneficial to provide improved compositions and admixtures having an improved flavor profile. Quercetin possesses significant physiological and medicinal benefits including: inhibition of pathological angiogenesis; stimulates physiological angiogenesis; improves endothelial health and vascular function; stimulates progenitor cells in regeneration; suppresses growth of cancer stem cells; has cancer chemopreventive effects; improves gut microbiome composition; improves metabolism; protects DNA against oxidative damage; reduces telomere attrition; acts as a senolytic and slows cellular aging; enhances immune function; exerts anti-inflammatory effects; causes adipocyte browning; increases adaptive thermogenesis; and reduces adipocyte accumulation.

Proanthocyanidins are a genus of plant-based polyphenols comprising oligomers/polymers of flavan-3-ols, such as, e.g., polymers of catechin, epicatechin, and epigallocatechin. Proanthocyanidins may range from flavanol dimers up to 11-mers.

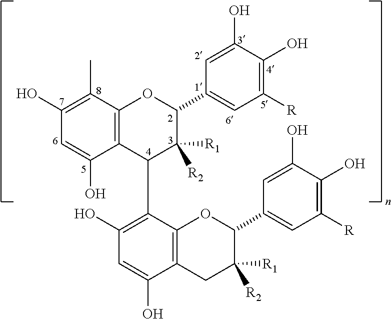

Proanthocyanidins are present in numerous plant sources including bark, flowers, fruits, nuts, and seeds, and are associated with numerous health benefits including antioxidant, anticancer, antidiabetic, antimicrobial, and neuroprotective properties. Proanthocyanidins have an astringent odor and flavor, and therefore it would be advantageous to develop improved compositions and admixtures having a more pleasant flavor profile. Proanthocyanidins possesses significant physiological and medicinal benefits including: inhibition of pathological angiogenesis; stimulates physiological angiogenesis; improves endothelial health and vascular function; stimulates progenitor cells in regeneration; suppresses growth of cancer stem cells; has cancer chemopreventive effects; improves gut microbiome composition; improves metabolism; protects DNA against oxidative damage; enhances immune function; exerts anti-inflammatory effects; causes adipocyte browning; increases adaptive thermogenesis; and reduces adipocyte accumulation.

Lactobacillus reuteri is a Bacillus bacteria that has been detected in breast milk and has been used in test and preclinical research. The strain L. reuteri 6475 has been demonstrated to increase bone density in mouse model. L. reuteri exerts significant physiological benefits including: stimulation of physiological angiogenesis; protects endothelial function; stimulates progenitor cells in tissue healing and regeneration; has cancer chemopreventive effects; improves gut microbiome composition; improves metabolism; suppresses obesity; promotes antioxidant activity to prevent oxidative damage to DNA; enhances immune function; exerts anti-inflammatory effects; causes adipocyte browning; increases adaptive thermogenesis; and reduces adipocyte accumulation. Akkermansia mucinophila is a mucin-degrading intestinal symbiont bacteria associated with digestive health. Ruminoccocus is a genus of anaerobic Gram-positive gut bacteria whose abundance has been correlated with lower incidences of conditions such as irritable bowel syndrome and Parkinson's disease.

Each of the twelve subject entities has known physiological benefits. However, in isolation their physiological potential, pharmacological interactions, and flavor and odor profiles are not optimal for support or improvement of physiological function. Combining a selection of these entities may mutually enhance their respective physiological benefits and may also alter and improve their flavor and odor profiles.

Selecting the ideal combination of these entities requires deep knowledge of the chemistry and pharmacology of each entity, and the sources in which they are present; the biological and pharmacological interactions between each entity and mammalian physiological defense systems; operational understanding of the mammalian physiological defense systems and how they relate to health and disease; data analytical techniques for assessing the mammalian physiological defense systems; the combinatorial properties of the entities; and end characteristics of the various combinations, such as, e.g., flavor and odor, solubility, stability, oral bioavailability, and others.

Thus, the present invention comprises compositions comprising a combination of a selection from twelve entities. In an embodiment, the present invention comprises any combination of any of: anthocyanin; β-(1-3,1-6)-D-glucan; chlorogenic acid; curcumin; epigallocatechin-3-gallate; genistein; hydroxytyrosol; Lactobacillus reuteri, Akkermansia mucinophila, and/or Ruminoccocus sp. bacteria; lycopene; omega-3 polyunsaturated fatty acid; quercetin; and proanthocyanidin. In a preferred embodiment, the composition comprises at least three of any of the above-named entities. In any embodiment, the composition may comprise at least four, at least five, at least six, at least seven, at least eight, at least nine, at least ten, at least eleven, or twelve of the above-named twelve entities.

More specifically, the composition may comprise at least anthocyanin; β-(1-3,1-6)-D-glucan; and chlorogenic acid. The composition may comprise at least anthocyanin; β-(1-3,1-6)-D-glucan; and curcumin. The composition may comprise at least anthocyanin; β-(1-3,1-6)-D-glucan; and epigallocatechin-3-gallate. The composition may comprise at least anthocyanin; β-(1-3,1-6)-D-glucan; and genistein. The composition may comprise at least anthocyanin; β-(1-3,1-6)-D-glucan; and hydroxytyrosol. The composition may comprise at least anthocyanin; β-(1-3,1-6)-D-glucan; and Lactobacillus reuteri, Akkermansia mucinophila, and/or Ruminoccocus sp. bacteria. The composition may comprise at least anthocyanin; β-(1-3,1-6)-D-glucan; and lycopene. The composition may comprise at least anthocyanin; β-(1-3,1-6)-D-glucan; and omega-3 polyunsaturated fatty acid. The composition may comprise at least anthocyanin; β-(1-3,1-6)-D-glucan; and quercetin. The composition may comprise at least anthocyanin; β-(1-3,1-6)-D-glucan; and proanthocyanidin.

The composition may comprise at least anthocyanin; chlorogenic acid; and curcumin. The composition may comprise at least anthocyanin; chlorogenic acid; and epigallocatechin-3-gallate. The composition may comprise at least anthocyanin; chlorogenic acid; and genistein. The composition may comprise at least anthocyanin; chlorogenic acid; and hydroxytyrosol. The composition may comprise at least anthocyanin; chlorogenic acid; and Lactobacillus reuteri, Akkermansia mucinophila, and/or Ruminoccocus sp. bacteria. The composition may comprise at least anthocyanin; chlorogenic acid; and lycopene. The composition may comprise at least anthocyanin; chlorogenic acid; and omega-3 polyunsaturated fatty acid. The composition may comprise at least anthocyanin; chlorogenic acid; and quercetin. The composition may comprise at least anthocyanin; chlorogenic acid; and proanthocyanidin.

The composition may comprise at least anthocyanin; curcumin; and epigallocatechin-3-gallate. The composition may comprise at least anthocyanin; curcumin; and genistein. The composition may comprise at least anthocyanin; curcumin; and hydroxytyrosol. The composition may comprise at least anthocyanin; curcumin; and Lactobacillus reuteri, Akkermansia mucinophila, and/or Ruminoccocus sp. bacteria. The composition may comprise at least anthocyanin; curcumin; and lycopene. The composition may comprise at least anthocyanin; curcumin; and omega-3 polyunsaturated fatty acid. The composition may comprise at least anthocyanin; curcumin; and quercetin. The composition may comprise at least anthocyanin; curcumin; and proanthocyanidin.

The composition may comprise at least anthocyanin; epigallocatechin-3-gallate; and genistein. The composition may comprise at least anthocyanin; epigallocatechin-3-gallate; and hydroxytyrosol. The composition may comprise at least anthocyanin; epigallocatechin-3-gallate; and Lactobacillus reuteri, Akkermansia mucinophila, and/or Ruminoccocus sp. bacteria. The composition may comprise at least anthocyanin; epigallocatechin-3-gallate; and lycopene. The composition may comprise at least anthocyanin; epigallocatechin-3-gallate; and omega-3 polyunsaturated fatty acid. The composition may comprise at least anthocyanin; epigallocatechin-3-gallate; and quercetin. The composition may comprise at least anthocyanin; epigallocatechin-3-gallate; and proanthocyanidin.

The composition may comprise at least anthocyanin; genistein; and hydroxytyrosol. The composition may comprise at least anthocyanin; genistein; and Lactobacillus reuteri, Akkermansia mucinophila, and/or Ruminoccocus sp. bacteria. The composition may comprise at least anthocyanin; genistein; and lycopene. The composition may comprise at least anthocyanin; genistein; and omega-3 polyunsaturated fatty acid. The composition may comprise at least anthocyanin; genistein; and quercetin. The composition may comprise at least anthocyanin; genistein; and proanthocyanidin.

The composition may comprise at least anthocyanin; hydroxytyrosol; and Lactobacillus reuteri, Akkermansia mucinophila, and/or Ruminoccocus sp. bacteria. The composition may comprise at least anthocyanin; hydroxytyrosol; and lycopene. The composition may comprise at least anthocyanin; hydroxytyrosol; and omega-3 polyunsaturated fatty acid. The composition may comprise at least anthocyanin; hydroxytyrosol; and quercetin. The composition may comprise at least anthocyanin; hydroxytyrosol; and proanthocyanidin.

The composition may comprise at least anthocyanin; Lactobacillus reuteri, Akkermansia mucinophila, and/or Ruminoccocus sp. bacteria; and lycopene. The composition may comprise at least anthocyanin; Lactobacillus reuteri, Akkermansia mucinophila, and/or Ruminoccocus sp. bacteria; and omega-3 polyunsaturated fatty acid. The composition may comprise at least anthocyanin; Lactobacillus reuteri, Akkermansia mucinophila, and/or Ruminoccocus sp. bacteria; and quercetin. The composition may comprise at least anthocyanin; Lactobacillus reuteri, Akkermansia mucinophila, and/or Ruminoccocus sp. bacteria; and proanthocyanidin.

The composition may comprise at least anthocyanin; lycopene; and omega-3 polyunsaturated fatty acid. The composition may comprise at least anthocyanin; lycopene; and quercetin. The composition may comprise at least anthocyanin; lycopene; and proanthocyanidin. The composition may comprise at least anthocyanin; omega-3 polyunsaturated fatty acid; and quercetin. The composition may comprise at least anthocyanin; omega-3 polyunsaturated fatty acid; and proanthocyanidin. The composition may comprise at least anthocyanin; quercetin; and proanthocyanidin.

The composition may comprise at least β-(1-3,1-6)-D-glucan; chlorogenic acid; and curcumin. The composition may comprise at least β-(1-3,1-6)-D-glucan; chlorogenic acid; and epigallocatechin-3-gallate. The composition may comprise at least β-(1-3,1-6)-D-glucan; chlorogenic acid; and genistein. The composition may comprise at least β-(1-3,1-6)-D-glucan; chlorogenic acid; and hydroxytyrosol. The composition may comprise at least β-(1-3,1-6)-D-glucan; chlorogenic acid; and Lactobacillus reuteri, Akkermansia mucinophila, and/or Ruminoccocus sp. bacteria. The composition may comprise at least β-(1-3,1-6)-D-glucan; chlorogenic acid; and lycopene. The composition may comprise at least β-(1-3,1-6)-D-glucan; chlorogenic acid; and omega-3 polyunsaturated fatty acid. The composition may comprise at least β-(1-3,1-6)-D-glucan; chlorogenic acid; and quercetin. The composition may comprise at least β-(1-3,1-6)-D-glucan; chlorogenic acid; and proanthocyanidin.

The composition may comprise at least β-(1-3,1-6)-D-glucan; curcumin; and epigallocatechin-3-gallate. The composition may comprise at least β-(1-3,1-6)-D-glucan; curcumin; and genistein. The composition may comprise at least β-(1-3,1-6)-D-glucan; curcumin; and hydroxytyrosol. The composition may comprise at least β-(1-3,1-6)-D-glucan; curcumin; and Lactobacillus reuteri, Akkermansia mucinophila, and/or Ruminoccocus sp. bacteria. The composition may comprise at least β-(1-3,1-6)-D-glucan; curcumin; and lycopene. The composition may comprise at least β-(1-3,1-6)-D-glucan; curcumin; and omega-3 polyunsaturated fatty acid. The composition may comprise at least β-(1-3,1-6)-D-glucan; curcumin; and quercetin. The composition may comprise at least β-(1-3,1-6)-D-glucan; curcumin; and proanthocyanidin.

The composition may comprise at least β-(1-3,1-6)-D-glucan; epigallocatechin-3-gallate; and genistein. The composition may comprise at least β-(1-3,1-6)-D-glucan; epigallocatechin-3-gallate; and hydroxytyrosol. The composition may comprise at least β-(1-3,1-6)-D-glucan; epigallocatechin-3-gallate; and Lactobacillus reuteri, Akkermansia mucinophila, and/or Ruminoccocus sp. bacteria. The composition may comprise at least β-(1-3,1-6)-D-glucan; epigallocatechin-3-gallate; and lycopene. The composition may comprise at least β-(1-3,1-6)-D-glucan; epigallocatechin-3-gallate; and omega-3 polyunsaturated fatty acid. The composition may comprise at least β-(1-3,1-6)-D-glucan; epigallocatechin-3-gallate; and quercetin. The composition may comprise at least β-(1-3,1-6)-D-glucan; epigallocatechin-3-gallate; and proanthocyanidin.

The composition may comprise at least β-(1-3,1-6)-D-glucan; genistein; and hydroxytyrosol. The composition may comprise at least β-(1-3,1-6)-D-glucan; genistein; and Lactobacillus reuteri, Akkermansia mucinophila, and/or Ruminoccocus sp. bacteria. The composition may comprise at least β-(1-3,1-6)-D-glucan; genistein; and lycopene. The composition may comprise at least β-(1-3,1-6)-D-glucan; genistein; and omega-3 polyunsaturated fatty acid. The composition may comprise at least β-(1-3,1-6)-D-glucan; genistein; and quercetin. The composition may comprise at least β-(1-3,1-6)-D-glucan; genistein; and proanthocyanidin. The composition may comprise at least β-(1-3,1-6)-D-glucan; hydroxytyrosol; and Lactobacillus reuteri, Akkermansia mucinophila, and/or Ruminoccocus sp. bacteria. The composition may comprise at least β-(1-3,1-6)-D-glucan; hydroxytyrosol; and lycopene. The composition may comprise at least β-(1-3,1-6)-D-glucan; hydroxytyrosol; and omega-3 polyunsaturated fatty acid. The composition may comprise at least β-(1-3,1-6)-D-glucan; hydroxytyrosol; and quercetin. The composition may comprise at least β-(1-3,1-6)-D-glucan; hydroxytyrosol; and proanthocyanidin.

The composition may comprise at least β-(1-3,1-6)-D-glucan; Lactobacillus reuteri, Akkermansia mucinophila, and/or Ruminoccocus sp. bacteria; and lycopene. The composition may comprise at least β-(1-3,1-6)-D-glucan; Lactobacillus reuteri, Akkermansia mucinophila, and/or Ruminoccocus sp. bacteria; and omega-3 polyunsaturated fatty acid. The composition may comprise at least β-(1-3,1-6)-D-glucan; Lactobacillus reuteri, Akkermansia mucinophila, and/or Ruminoccocus sp. bacteria; and quercetin. The composition may comprise at least β-(1-3,1-6)-D-glucan; Lactobacillus reuteri, Akkermansia mucinophila, and/or Ruminoccocus sp. bacteria; and proanthocyanidin.

The composition may comprise at least β-(1-3,1-6)-D-glucan; lycopene; and omega-3 polyunsaturated fatty acid. The composition may comprise at least β-(1-3,1-6)-D-glucan; lycopene; and quercetin. The composition may comprise at least β-(1-3,1-6)-D-glucan; lycopene; and proanthocyanidin. The composition may comprise at least β-(1-3,1-6)-D-glucan; omega-3 polyunsaturated fatty acid; and quercetin. The composition may comprise at least β-(1-3,1-6)-D-glucan; omega-3 polyunsaturated fatty acid; and proanthocyanidin.

The composition may comprise at least β-(1-3,1-6)-D-glucan; quercetin; and proanthocyanidin. The composition may comprise at least chlorogenic acid; curcumin; and epigallocatechin-3-gallate. The composition may comprise at least chlorogenic acid; curcumin; and genistein. The composition may comprise at least chlorogenic acid; curcumin; and hydroxytyrosol. The composition may comprise at least chlorogenic acid; curcumin; and Lactobacillus reuteri, Akkermansia mucinophila, and/or Ruminoccocus sp. bacteria. The composition may comprise at least chlorogenic acid; curcumin; and lycopene. The composition may comprise at least chlorogenic acid; curcumin; and omega-3 polyunsaturated fatty acid. The composition may comprise at least chlorogenic acid; curcumin; and quercetin. The composition may comprise at least chlorogenic acid; curcumin; and proanthocyanidin.

The composition may comprise at least chlorogenic acid; epigallocatechin-3-gallate; and genistein. The composition may comprise at least chlorogenic acid; epigallocatechin-3-gallate; and hydroxytyrosol. The composition may comprise at least chlorogenic acid; epigallocatechin-3-gallate; and Lactobacillus reuteri, Akkermansia mucinophila, and/or Ruminoccocus sp. bacteria. The composition may comprise at least chlorogenic acid; epigallocatechin-3-gallate; and lycopene. The composition may comprise at least chlorogenic acid; epigallocatechin-3-gallate; and omega-3 polyunsaturated fatty acid. The composition may comprise at least chlorogenic acid; epigallocatechin-3-gallate; and quercetin. The composition may comprise at least chlorogenic acid; epigallocatechin-3-gallate; and proanthocyanidin.

The composition may comprise at least chlorogenic acid; genistein; and hydroxytyrosol. The composition may comprise at least chlorogenic acid; genistein; and Lactobacillus reuteri, Akkermansia mucinophila, and/or Ruminoccocus sp. bacteria. The composition may comprise at least chlorogenic acid; genistein; and lycopene. The composition may comprise at least chlorogenic acid; genistein; and omega-3 polyunsaturated fatty acid. The composition may comprise at least chlorogenic acid; genistein; and quercetin. The composition may comprise at least chlorogenic acid; genistein; and proanthocyanidin. The composition may comprise at least chlorogenic acid; hydroxytyrosol; and Lactobacillus reuteri, Akkermansia mucinophila, and/or Ruminoccocus sp. bacteria. The composition may comprise at least chlorogenic acid; hydroxytyrosol; and lycopene. The composition may comprise at least chlorogenic acid; hydroxytyrosol; and omega-3 polyunsaturated fatty acid. The composition may comprise at least chlorogenic acid; hydroxytyrosol; and quercetin. The composition may comprise at least chlorogenic acid; hydroxytyrosol; and proanthocyanidin.

The composition may comprise at least chlorogenic acid; Lactobacillus reuteri, Akkermansia mucinophila, and/or Ruminoccocus sp. bacteria; and lycopene. The composition may comprise at least chlorogenic acid; Lactobacillus reuteri, Akkermansia mucinophila, and/or Ruminoccocus sp. bacteria; and omega-3 polyunsaturated fatty acid. The composition may comprise at least chlorogenic acid; Lactobacillus reuteri, Akkermansia mucinophila, and/or Ruminoccocus sp. bacteria; and quercetin. The composition may comprise at least chlorogenic acid; Lactobacillus reuteri, Akkermansia mucinophila, and/or Ruminoccocus sp. bacteria; and proanthocyanidin.

The composition may comprise at least chlorogenic acid; lycopene; and omega-3 polyunsaturated fatty acid. The composition may comprise at least chlorogenic acid; lycopene; and quercetin. The composition may comprise at least chlorogenic acid; lycopene; and proanthocyanidin. The composition may comprise at least chlorogenic acid; omega-3 polyunsaturated fatty acid; and quercetin. The composition may comprise at least chlorogenic acid; omega-3 polyunsaturated fatty acid; and proanthocyanidin. The composition may comprise at least chlorogenic acid; quercetin; and proanthocyanidin.

The composition may comprise at least curcumin; epigallocatechin-3-gallate; and genistein. The composition may comprise at least curcumin; epigallocatechin-3-gallate; and hydroxytyrosol. The composition may comprise at least curcumin; epigallocatechin-3-gallate; and Lactobacillus reuteri, Akkermansia mucinophila, and/or Ruminoccocus sp. bacteria. The composition may comprise at least curcumin; epigallocatechin-3-gallate; and lycopene. The composition may comprise at least curcumin; epigallocatechin-3-gallate; and omega-3 polyunsaturated fatty acid. The composition may comprise at least curcumin; epigallocatechin-3-gallate; and quercetin. The composition may comprise at least curcumin; epigallocatechin-3-gallate; and proanthocyanidin.

The composition may comprise at least curcumin; genistein; and hydroxytyrosol. The composition may comprise at least curcumin; genistein; and Lactobacillus reuteri, Akkermansia mucinophila, and/or Ruminoccocus sp. bacteria. The composition may comprise at least curcumin; genistein; and lycopene. The composition may comprise at least curcumin; genistein; and omega-3 polyunsaturated fatty acid. The composition may comprise at least curcumin; genistein; and quercetin. The composition may comprise at least curcumin; genistein; and proanthocyanidin.

The composition may comprise at least curcumin; hydroxytyrosol; and Lactobacillus reuteri, Akkermansia mucinophila, and/or Ruminoccocus sp. bacteria. The composition may comprise at least curcumin; hydroxytyrosol; and lycopene. The composition may comprise at least curcumin; hydroxytyrosol; and omega-3 polyunsaturated fatty acid. The composition may comprise at least curcumin; hydroxytyrosol; and quercetin. The composition may comprise at least curcumin; hydroxytyrosol; and proanthocyanidin.

The composition may comprise at least curcumin; Lactobacillus reuteri, Akkermansia mucinophila, and/or Ruminoccocus sp. bacteria; and lycopene. The composition may comprise at least curcumin; Lactobacillus reuteri, Akkermansia mucinophila, and/or Ruminoccocus sp. bacteria; and omega-3 polyunsaturated fatty acid. The composition may comprise at least curcumin; Lactobacillus reuteri, Akkermansia mucinophila, and/or Ruminoccocus sp. bacteria; and quercetin. The composition may comprise at least curcumin; Lactobacillus reuteri, Akkermansia mucinophila, and/or Ruminoccocus sp. bacteria; and proanthocyanidin.

The composition may comprise at least curcumin; lycopene; and omega-3 polyunsaturated fatty acid. The composition may comprise at least curcumin; lycopene; and quercetin. The composition may comprise at least curcumin; lycopene; and proanthocyanidin. The composition may comprise at least curcumin; omega-3 polyunsaturated fatty acid; and quercetin. The composition may comprise at least curcumin; omega-3 polyunsaturated fatty acid; and proanthocyanidin. The composition may comprise at least curcumin; quercetin; and proanthocyanidin.

The composition may comprise at least epigallocatechin-3 gallate; genistein; and hydroxytyrosol. The composition may comprise at least epigallocatechin-3 gallate; genistein; and Lactobacillus reuteri, Akkermansia mucinophila, and/or Ruminoccocus sp. bacteria. The composition may comprise at least epigallocatechin-3 gallate; genistein; and lycopene. The composition may comprise at least epigallocatechin-3 gallate; genistein; and omega-3 polyunsaturated fatty acid. The composition may comprise at least epigallocatechin-3 gallate; genistein; and quercetin. The composition may comprise at least epigallocatechin-3 gallate; genistein; and proanthocyanidin.

The composition may comprise at least epigallocatechin-3 gallate; hydroxytyrosol; and Lactobacillus reuteri, Akkermansia mucinophila, and/or Ruminoccocus sp. bacteria. The composition may comprise at least epigallocatechin-3 gallate; hydroxytyrosol; and lycopene. The composition may comprise at least epigallocatechin-3 gallate; hydroxytyrosol; and omega-3 polyunsaturated fatty acid. The composition may comprise at least epigallocatechin-3 gallate; hydroxytyrosol; and quercetin. The composition may comprise at least epigallocatechin-3 gallate; hydroxytyrosol; and proanthocyanidin. The composition may comprise at least epigallocatechin-3 gallate; Lactobacillus reuteri, Akkermansia mucinophila, and/or Ruminoccocus sp. bacteria; and lycopene. The composition may comprise at least epigallocatechin-3 gallate; Lactobacillus reuteri, Akkermansia mucinophila, and/or Ruminoccocus sp. bacteria; and omega-3 polyunsaturated fatty acid. The composition may comprise at least epigallocatechin-3 gallate; Lactobacillus reuteri, Akkermansia mucinophila, and/or Ruminoccocus sp. bacteria; and quercetin. The composition may comprise at least epigallocatechin-3 gallate; Lactobacillus reuteri, Akkermansia mucinophila, and/or Ruminoccocus sp. bacteria; and proanthocyanidin.

The composition may comprise at least epigallocatechin-3 gallate; lycopene; and omega-3 polyunsaturated fatty acid. The composition may comprise at least epigallocatechin-3 gallate; lycopene; and quercetin. The composition may comprise at least epigallocatechin-3 gallate; lycopene; and proanthocyanidin. The composition may comprise at least epigallocatechin-3 gallate; omega-3 polyunsaturated fatty acid; and quercetin. The composition may comprise at least epigallocatechin-3 gallate; omega-3 polyunsaturated fatty acid; and proanthocyanidin. The composition may comprise at least epigallocatechin-3 gallate; quercetin; and proanthocyanidin.

The composition may comprise at least genistein; hydroxytyrosol; and Lactobacillus reuteri, Akkermansia mucinophila, and/or Ruminoccocus sp. bacteria. The composition may comprise at least genistein; hydroxytyrosol; and lycopene. The composition may comprise at least genistein; hydroxytyrosol; and omega-3 polyunsaturated fatty acid. The composition may comprise at least genistein; hydroxytyrosol; and quercetin. The composition may comprise at least genistein; hydroxytyrosol; and proanthocyanidin. The composition may comprise at least genistein; Lactobacillus reuteri, Akkermansia mucinophila, and/or Ruminoccocus sp. bacteria; and lycopene. The composition may comprise at least genistein; Lactobacillus reuteri, Akkermansia mucinophila, and/or Ruminoccocus sp. bacteria; and omega-3 polyunsaturated fatty acid. The composition may comprise at least genistein; Lactobacillus reuteri, Akkermansia mucinophila, and/or Ruminoccocus sp. bacteria; and quercetin. The composition may comprise at least genistein; Lactobacillus reuteri, Akkermansia mucinophila, and/or Ruminoccocus sp. bacteria; and proanthocyanidin.

The composition may comprise at least genistein; lycopene; and omega-3 polyunsaturated fatty acid. The composition may comprise at least genistein; lycopene; and quercetin. The composition may comprise at least genistein; lycopene; and proanthocyanidin. The composition may comprise at least genistein; omega-3 polyunsaturated fatty acid; and quercetin. The composition may comprise at least genistein; omega-3 polyunsaturated fatty acid; and proanthocyanidin. The composition may comprise at least genistein; quercetin; and proanthocyanidin.

The composition may comprise at least hydroxytyrosol; Lactobacillus reuteri, Akkermansia mucinophila, and/or Ruminoccocus sp. bacteria; and lycopene. The composition may comprise at least hydroxytyrosol; Lactobacillus reuteri, Akkermansia mucinophila, and/or Ruminoccocus sp. bacteria; and omega-3 polyunsaturated fatty acid. The composition may comprise at least hydroxytyrosol; Lactobacillus reuteri, Akkermansia mucinophila, and/or Ruminoccocus sp. bacteria; and quercetin. The composition may comprise at least hydroxytyrosol; Lactobacillus reuteri, Akkermansia mucinophila, and/or Ruminoccocus sp. bacteria; and proanthocyanidin.

The composition may comprise at least hydroxytyrosol; lycopene; and omega-3 polyunsaturated fatty acid. The composition may comprise at least hydroxytyrosol; lycopene; and quercetin. The composition may comprise at least hydroxytyrosol; lycopene; and proanthocyanidin. The composition may comprise at least hydroxytyrosol; omega-3 polyunsaturated fatty acid; and quercetin. The composition may comprise at least hydroxytyrosol; omega-3 polyunsaturated fatty acid; and proanthocyanidin. The composition may comprise at least hydroxytyrosol; quercetin; and proanthocyanidin.

The composition may comprise at least Lactobacillus reuteri, Akkermansia mucinophila, and/or Ruminoccocus sp. bacteria; lycopene; and omega-3 polyunsaturated fatty acid. The composition may comprise at least Lactobacillus reuteri, Akkermansia mucinophila, and/or Ruminoccocus sp. bacteria; lycopene; and quercetin. The composition may comprise at least Lactobacillus reuteri, Akkermansia mucinophila, and/or Ruminoccocus sp. bacteria; lycopene; and proanthocyanidin. The composition may comprise at least Lactobacillus reuteri, Akkermansia mucinophila, and/or Ruminoccocus sp. bacteria; omega-3 polyunsaturated fatty acid; and quercetin. The composition may comprise at least Lactobacillus reuteri, Akkermansia mucinophila, and/or Ruminoccocus sp. bacteria; omega-3 polyunsaturated fatty acid; and proanthocyanidin. The composition may comprise at least Lactobacillus reuteri, Akkermansia mucinophila, and/or Ruminoccocus sp. bacteria; quercetin; and proanthocyanidin. The composition may comprise at least lycopene; omega-3 polyunsaturated fatty acid; and quercetin. The composition may comprise at least lycopene; omega-3 polyunsaturated fatty acid; and proanthocyanidin. The composition may comprise at least omega-3 polyunsaturated fatty acid; quercetin and proanthocyanidin.

In any aspect or embodiment, the composition may further comprise spices (e.g., cinnamon spice, nutmeg, cumin, clove, turmeric, anise, ginger, cardamom, chili, black pepper, Szechuan pepper, paprika, mustard, etc.). In any aspect or embodiment, the composition may further comprise a food product, beverage product, or both. In any aspect or embodiment, the composition may further comprise flavorings, spices, an oil pack, a meal replacement, a functional beverage, a functional food, a medical food, condiments, prescription dietary food, pet food, or any combination thereof. In any aspect or embodiment, the composition may comprise a dietary supplement. In any aspect or embodiment, the composition and method of the present invention may comprise dietary sustenance, foods, and/or supplements for human and/or animal space travel, space station settlement, and extraterrestrial settlement.

In any aspect or embodiment, the composition may be selected for its odor profile, flavor profile, or both. In any aspect or embodiment, the composition may be selected for its solubility. In any aspect or embodiment, the composition may be selected for its stability. In any aspect or embodiment, the composition may be selected for its potency. In any aspect or embodiment, the composition may be selected for its bioavailability. In any aspect or embodiment, the composition may be selected for its flavor profile.

In any aspect or embodiment, the lycopene may comprise all-trans-lycopene, or a mixture of trans- and cis-lycopenes. In any aspect or embodiment, the omega-3 polyunsaturated fatty acids may be derived from marine organisms, including, e.g., fish. In any aspect or embodiment, the Lactobacillus reuteri may be the strain L. reuteri ATCC PTA 6475.

In any aspect or embodiment, the composition may further comprise an acceptable carrier, excipient, buffer, or any combination thereof.

In any aspect or embodiment, the composition may be delivered in the form of a pill, capsule, cream, salve, syrup, dermal patch, dissolvable strip (e.g., to be dissolved on a subject's tongue), suppository, intravenous drip, aqueous solution, non-aqueous solution, powder (e.g., to be dissolved in aqueous solvent), granules, or any combination thereof. In any aspect or embodiment, the composition may be in aqueous solution. In any aspect or embodiment, the composition may be prepackaged in a container, envelope, canister, pack, or syringe. In any aspect or embodiment, the composition may further comprise instructions for administration. In any aspect or embodiment, the composition may be delivered to a mammalian subject by ingestion, intravenous injection, peritoneal injection, muscular injection, nasally, orally, sublingually, rectally, by inhalation or any combination thereof. In any aspect or embodiment, the components of the composition are selected in biologically effective amounts and/or concentrations.

The twelve subject entities, alone or in combination, can be combined with pharmaceutically acceptable carriers, diluents, excipients and reagents useful in preparing a formulation that is generally safe, non-toxic, and desirable, and includes excipients that are acceptable for mammalian, e.g., human or primate, use. Such excipients can be solid, liquid, semisolid, or, in the case of an aerosol composition, gaseous. Examples of such carriers, diluents and excipients include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerin, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates; detergents such as Tween® 20 to prevent aggregation; and compounds for the adjustment of tonicity such as sodium chloride or dextrose. The pH can be adjusted with acids or bases, such as hydrochloric acid or sodium hydroxide. In particular embodiments, the pharmaceutical compositions are sterile.

Pharmaceutical compositions may further include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In some cases, the composition is sterile and should be fluid such that it can be drawn into a syringe or delivered to a subject from a syringe. In certain embodiments, it is stable under the conditions of manufacture and storage and is preserved against the contaminating action of microorganisms such as bacteria and fungi. The carrier can be, e.g., a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the composition of the present disclosure in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, the pharmaceutical compositions are prepared with carriers that will protect the antibody or antigen-binding fragment thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers. These can be prepared according to methods known to those skilled in the art.

It may be advantageous to formulate the pharmaceutical compositions in dosage unit form for ease of administration and uniformity of dosage. Dosage unit form as used herein refers to physically discrete units suited as unitary dosages for the subject to be treated; each unit containing a predetermined quantity of active antibody or antigen-binding fragment thereof calculated to produce the desired therapeutic effect in association with the required pharmaceutical carrier. The specification for the dosage unit forms are dictated by and directly dependent on the unique characteristics of the antibody or antigen-binding fragment thereof and the particular therapeutic effect to be achieved, and the limitations inherent in the art of compounding such an active antibody or antigen-binding fragment thereof for the treatment of individuals.

The pharmaceutical compositions can be included in a container, pack, or dispenser, e.g., syringe, e.g., a prefilled syringe, together with instructions for administration.

The pharmaceutical compositions of the present disclosure may be delivered to a subject in the form of a pill, capsule, cream, salve, syrup, dermal patch, suppository, intravenous drip, aqueous solution, non-aqueous solution, or any combination of thereof.

The pharmaceutical compositions of the present disclosure may be delivered to a subject by direct ophthalmic application, muscular injection, intravenous injection, peritoneal injection, nasally, orally, rectally, or any combination thereof.

In another aspect, the present invention comprises a method of providing compositions comprising a combination of a selection from twelve entities. In an embodiment, the present invention comprises a method of providing any combination of any of: anthocyanin; β-(1-3,1-6)-D-glucan; chlorogenic acid; curcumin; epigallocatechin-3-gallate; genistein; hydroxytyrosol; Lactobacillus reuteri, Akkermansia mucinophila, and/or Ruminoccocus sp. bacteria; lycopene; omega-3 polyunsaturated fatty acid; quercetin; and proanthocyanidin. In a preferred embodiment, the provided composition comprises at least three of any of the above-named entities. In any embodiment, the provided composition may comprise at least four, at least five, at least six, at least seven, at least eight, at least nine, at least ten, at least eleven, or twelve of the above-named twelve entities.

More specifically, the provided composition may comprise at least anthocyanin; β-(1-3,1-6)-D-glucan; and chlorogenic acid. The provided composition may comprise at least anthocyanin; β-(1-3,1-6)-D-glucan; and curcumin. The provided composition may comprise at least anthocyanin; β-(1-3,1-6)-D-glucan; and epigallocatechin-3-gallate. The provided composition may comprise at least anthocyanin; β-(1-3,1-6)-D-glucan; and genistein. The provided composition may comprise at least anthocyanin; β-(1-3,1-6)-D-glucan; and hydroxytyrosol. The provided composition may comprise at least anthocyanin; β-(1-3,1-6)-D-glucan; and Lactobacillus reuteri, Akkermansia mucinophila, and/or Ruminoccocus sp. bacteria. The provided composition may comprise at least anthocyanin; β-(1-3,1-6)-D-glucan; and lycopene. The provided composition may comprise at least anthocyanin; β-(1-3,1-6)-D-glucan; and omega-3 polyunsaturated fatty acid. The provided composition may comprise at least anthocyanin; β-(1-3,1-6)-D-glucan; and quercetin. The provided composition may comprise at least anthocyanin; β-(1-3,1-6)-D-glucan; and proanthocyanidin.

The provided composition may comprise at least anthocyanin; chlorogenic acid; and curcumin. The provided composition may comprise at least anthocyanin; chlorogenic acid; and epigallocatechin-3-gallate. The provided composition may comprise at least anthocyanin; chlorogenic acid; and genistein. The provided composition may comprise at least anthocyanin; chlorogenic acid; and hydroxytyrosol. The provided composition may comprise at least anthocyanin; chlorogenic acid; and Lactobacillus reuteri, Akkermansia mucinophila, and/or Ruminoccocus sp. bacteria. The provided composition may comprise at least anthocyanin; chlorogenic acid; and lycopene. The provided composition may comprise at least anthocyanin; chlorogenic acid; and omega-3 polyunsaturated fatty acid. The provided composition may comprise at least anthocyanin; chlorogenic acid; and quercetin. The provided composition may comprise at least anthocyanin; chlorogenic acid; and proanthocyanidin.

The provided composition may comprise at least anthocyanin; curcumin; and epigallocatechin-3-gallate. The provided composition may comprise at least anthocyanin; curcumin; and genistein. The provided composition may comprise at least anthocyanin; curcumin; and hydroxytyrosol. The provided composition may comprise at least anthocyanin; curcumin; and Lactobacillus reuteri, Akkermansia mucinophila, and/or Ruminoccocus sp. bacteria. The provided composition may comprise at least anthocyanin; curcumin; and lycopene. The provided composition may comprise at least anthocyanin; curcumin; and omega-3 polyunsaturated fatty acid. The provided composition may comprise at least anthocyanin; curcumin; and quercetin. The provided composition may comprise at least anthocyanin; curcumin; and proanthocyanidin.

The provided composition may comprise at least anthocyanin; epigallocatechin-3-gallate; and genistein. The provided composition may comprise at least anthocyanin; epigallocatechin-3-gallate; and hydroxytyrosol. The provided composition may comprise at least anthocyanin; epigallocatechin-3-gallate; and Lactobacillus reuteri, Akkermansia mucinophila, and/or Ruminoccocus sp. bacteria. The provided composition may comprise at least anthocyanin; epigallocatechin-3-gallate; and lycopene. The provided composition may comprise at least anthocyanin; epigallocatechin-3-gallate; and omega-3 polyunsaturated fatty acid. The provided composition may comprise at least anthocyanin; epigallocatechin-3-gallate; and quercetin. The provided composition may comprise at least anthocyanin; epigallocatechin-3-gallate; and proanthocyanidin.

The provided composition may comprise at least anthocyanin; genistein; and hydroxytyrosol. The provided composition may comprise at least anthocyanin; genistein; and Lactobacillus reuteri, Akkermansia mucinophila, and/or Ruminoccocus sp. bacteria. The provided composition may comprise at least anthocyanin; genistein; and lycopene. The provided composition may comprise at least anthocyanin; genistein; and omega-3 polyunsaturated fatty acid. The provided composition may comprise at least anthocyanin; genistein; and quercetin. The provided composition may comprise at least anthocyanin; genistein; and proanthocyanidin.

The provided composition may comprise at least anthocyanin; hydroxytyrosol; and Lactobacillus reuteri, Akkermansia mucinophila, and/or Ruminoccocus sp. bacteria. The provided composition may comprise at least anthocyanin; hydroxytyrosol; and lycopene. The provided composition may comprise at least anthocyanin; hydroxytyrosol; and omega-3 polyunsaturated fatty acid. The provided composition may comprise at least anthocyanin; hydroxytyrosol; and quercetin. The provided composition may comprise at least anthocyanin; hydroxytyrosol; and proanthocyanidin.

The provided composition may comprise at least anthocyanin; Lactobacillus reuteri, Akkermansia mucinophila, and/or Ruminoccocus sp. bacteria; and lycopene. The provided composition may comprise at least anthocyanin; Lactobacillus reuteri, Akkermansia mucinophila, and/or Ruminoccocus sp. bacteria; and omega-3 polyunsaturated fatty acid. The provided composition may comprise at least anthocyanin; Lactobacillus reuteri, Akkermansia mucinophila, and/or Ruminoccocus sp. bacteria; and quercetin. The provided composition may comprise at least anthocyanin; Lactobacillus reuteri, Akkermansia mucinophila, and/or Ruminoccocus sp. bacteria; and proanthocyanidin.

The provided composition may comprise at least anthocyanin; lycopene; and omega-3 polyunsaturated fatty acid. The provided composition may comprise at least anthocyanin; lycopene; and quercetin. The provided composition may comprise at least anthocyanin; lycopene; and proanthocyanidin. The provided composition may comprise at least anthocyanin; omega-3 polyunsaturated fatty acid; and quercetin. The provided composition may comprise at least anthocyanin; omega-3 polyunsaturated fatty acid; and proanthocyanidin. The provided composition may comprise at least anthocyanin; quercetin; and proanthocyanidin.

The provided composition may comprise at least β-(1-3,1-6)-D-glucan; chlorogenic acid; and curcumin. The provided composition may comprise at least β-(1-3,1-6)-D-glucan; chlorogenic acid; and epigallocatechin-3-gallate. The provided composition may comprise at least β-(1-3,1-6)-D-glucan; chlorogenic acid; and genistein. The provided composition may comprise at least β-(1-3,1-6)-D-glucan; chlorogenic acid; and hydroxytyrosol. The provided composition may comprise at least β-(1-3,1-6)-D-glucan; chlorogenic acid; and Lactobacillus reuteri, Akkermansia mucinophila, and/or Ruminoccocus sp. bacteria. The provided composition may comprise at least β-(1-3,1-6)-D-glucan; chlorogenic acid; and lycopene. The provided composition may comprise at least β-(1-3,1-6)-D-glucan; chlorogenic acid; and omega-3 polyunsaturated fatty acid. The provided composition may comprise at least β-(1-3,1-6)-D-glucan; chlorogenic acid; and quercetin. The provided composition may comprise at least β-(1-3,1-6)-D-glucan; chlorogenic acid; and proanthocyanidin.

The provided composition may comprise at least β-(1-3,1-6)-D-glucan; curcumin; and epigallocatechin-3-gallate. The provided composition may comprise at least β-(1-3,1-6)-D-glucan; curcumin; and genistein. The provided composition may comprise at least β-(1-3,1-6)-D-glucan; curcumin; and hydroxytyrosol. The provided composition may comprise at least β-(1-3,1-6)-D-glucan; curcumin; and Lactobacillus reuteri, Akkermansia mucinophila, and/or Ruminoccocus sp. bacteria. The provided composition may comprise at least β-(1-3,1-6)-D-glucan; curcumin; and lycopene. The provided composition may comprise at least β-(1-3,1-6)-D-glucan; curcumin; and omega-3 polyunsaturated fatty acid. The provided composition may comprise at least β-(1-3,1-6)-D-glucan; curcumin; and quercetin. The provided composition may comprise at least β-(1-3,1-6)-D-glucan; curcumin; and proanthocyanidin.

The provided composition may comprise at least β-(1-3,1-6)-D-glucan; epigallocatechin-3-gallate; and genistein. The provided composition may comprise at least β-(1-3,1-6)-D-glucan; epigallocatechin-3-gallate; and hydroxytyrosol. The provided composition may comprise at least β-(1-3,1-6)-D-glucan; epigallocatechin-3-gallate; and Lactobacillus reuteri, Akkermansia mucinophila, and/or Ruminoccocus sp. bacteria. The provided composition may comprise at least β-(1-3,1-6)-D-glucan; epigallocatechin-3-gallate; and lycopene. The provided composition may comprise at least β-(1-3,1-6)-D-glucan; epigallocatechin-3-gallate; and omega-3 polyunsaturated fatty acid. The provided composition may comprise at least β-(1-3,1-6)-D-glucan; epigallocatechin-3-gallate; and quercetin. The provided composition may comprise at least β-(1-3,1-6)-D-glucan; epigallocatechin-3-gallate; and proanthocyanidin.

The provided composition may comprise at least β-(1-3,1-6)-D-glucan; genistein; and hydroxytyrosol. The provided composition may comprise at least β-(1-3,1-6)-D-glucan; genistein; and Lactobacillus reuteri, Akkermansia mucinophila, and/or Ruminoccocus sp. bacteria. The provided composition may comprise at least β-(1-3,1-6)-D-glucan; genistein; and lycopene. The provided composition may comprise at least β-(1-3,1-6)-D-glucan; genistein; and omega-3 polyunsaturated fatty acid. The provided composition may comprise at least β-(1-3,1-6)-D-glucan; genistein; and quercetin. The provided composition may comprise at least β-(1-3,1-6)-D-glucan; genistein; and proanthocyanidin. The provided composition may comprise at least β-(1-3,1-6)-D-glucan; hydroxytyrosol; and Lactobacillus reuteri, Akkermansia mucinophila, and/or Ruminoccocus sp. bacteria. The provided composition may comprise at least β-(1-3,1-6)-D-glucan; hydroxytyrosol; and lycopene. The provided composition may comprise at least β-(1-3,1-6)-D-glucan; hydroxytyrosol; and omega-3 polyunsaturated fatty acid. The provided composition may comprise at least β-(1-3,1-6)-D-glucan; hydroxytyrosol; and quercetin. The provided composition may comprise at least β-(1-3,1-6)-D-glucan; hydroxytyrosol; and proanthocyanidin.

The provided composition may comprise at least β-(1-3,1-6)-D-glucan; Lactobacillus reuteri, Akkermansia mucinophila, and/or Ruminoccocus sp. bacteria; and lycopene. The provided composition may comprise at least β-(1-3,1-6)-D-glucan; Lactobacillus reuteri, Akkermansia mucinophila, and/or Ruminoccocus sp. bacteria; and omega-3 polyunsaturated fatty acid. The provided composition may comprise at least β-(1-3,1-6)-D-glucan; Lactobacillus reuteri, Akkermansia mucinophila, and/or Ruminoccocus sp. bacteria; and quercetin. The provided composition may comprise at least β-(1-3,1-6)-D-glucan; Lactobacillus reuteri, Akkermansia mucinophila, and/or Ruminoccocus sp. bacteria; and proanthocyanidin.

The provided composition may comprise at least β-(1-3,1-6)-D-glucan; lycopene; and omega-3 polyunsaturated fatty acid. The provided composition may comprise at least β-(1-3,1-6)-D-glucan; lycopene; and quercetin. The provided composition may comprise at least β-(1-3,1-6)-D-glucan; lycopene; and proanthocyanidin. The provided composition may comprise at least β-(1-3,1-6)-D-glucan; omega-3 polyunsaturated fatty acid; and quercetin. The provided composition may comprise at least β-(1-3,1-6)-D-glucan; omega-3 polyunsaturated fatty acid; and proanthocyanidin.

The provided composition may comprise at least β-(1-3,1-6)-D-glucan; quercetin; and proanthocyanidin. The provided composition may comprise at least chlorogenic acid; curcumin; and epigallocatechin-3-gallate. The provided composition may comprise at least chlorogenic acid; curcumin; and genistein. The provided composition may comprise at least chlorogenic acid; curcumin; and hydroxytyrosol. The provided composition may comprise at least chlorogenic acid; curcumin; and Lactobacillus reuteri, Akkermansia mucinophila, and/or Ruminoccocus sp. bacteria. The provided composition may comprise at least chlorogenic acid; curcumin; and lycopene. The provided composition may comprise at least chlorogenic acid; curcumin; and omega-3 polyunsaturated fatty acid. The provided composition may comprise at least chlorogenic acid; curcumin; and quercetin. The provided composition may comprise at least chlorogenic acid; curcumin; and proanthocyanidin.

The provided composition may comprise at least chlorogenic acid; epigallocatechin-3-gallate; and genistein. The provided composition may comprise at least chlorogenic acid; epigallocatechin-3-gallate; and hydroxytyrosol. The provided composition may comprise at least chlorogenic acid; epigallocatechin-3-gallate; and Lactobacillus reuteri, Akkermansia mucinophila, and/or Ruminoccocus sp. bacteria. The provided composition may comprise at least chlorogenic acid; epigallocatechin-3-gallate; and lycopene. The provided composition may comprise at least chlorogenic acid; epigallocatechin-3-gallate; and omega-3 polyunsaturated fatty acid. The provided composition may comprise at least chlorogenic acid; epigallocatechin-3-gallate; and quercetin. The provided composition may comprise at least chlorogenic acid; epigallocatechin-3-gallate; and proanthocyanidin.

The provided composition may comprise at least chlorogenic acid; genistein; and hydroxytyrosol. The provided composition may comprise at least chlorogenic acid; genistein; and Lactobacillus reuteri, Akkermansia mucinophila, and/or Ruminoccocus sp. bacteria. The provided composition may comprise at least chlorogenic acid; genistein; and lycopene. The provided composition may comprise at least chlorogenic acid; genistein; and omega-3 polyunsaturated fatty acid. The provided composition may comprise at least chlorogenic acid; genistein; and quercetin. The provided composition may comprise at least chlorogenic acid; genistein; and proanthocyanidin. The provided composition may comprise at least chlorogenic acid; hydroxytyrosol; and Lactobacillus reuteri, Akkermansia mucinophila, and/or Ruminoccocus sp. bacteria. The provided composition may comprise at least chlorogenic acid; hydroxytyrosol; and lycopene. The provided composition may comprise at least chlorogenic acid; hydroxytyrosol; and omega-3 polyunsaturated fatty acid. The provided composition may comprise at least chlorogenic acid; hydroxytyrosol; and quercetin. The provided composition may comprise at least chlorogenic acid; hydroxytyrosol; and proanthocyanidin.

The provided composition may comprise at least chlorogenic acid; Lactobacillus reuteri, Akkermansia mucinophila, and/or Ruminoccocus sp. bacteria; and lycopene. The provided composition may comprise at least chlorogenic acid; Lactobacillus reuteri, Akkermansia mucinophila, and/or Ruminoccocus sp. bacteria; and omega-3 polyunsaturated fatty acid. The provided composition may comprise at least chlorogenic acid; Lactobacillus reuteri, Akkermansia mucinophila, and/or Ruminoccocus sp. bacteria; and quercetin. The provided composition may comprise at least chlorogenic acid; Lactobacillus reuteri, Akkermansia mucinophila, and/or Ruminoccocus sp. bacteria; and proanthocyanidin.

The provided composition may comprise at least chlorogenic acid; lycopene; and omega-3 polyunsaturated fatty acid. The provided composition may comprise at least chlorogenic acid; lycopene; and quercetin. The provided composition may comprise at least chlorogenic acid; lycopene; and proanthocyanidin. The provided composition may comprise at least chlorogenic acid; omega-3 polyunsaturated fatty acid; and quercetin. The provided composition may comprise at least chlorogenic acid; omega-3 polyunsaturated fatty acid; and proanthocyanidin. The provided composition may comprise at least chlorogenic acid; quercetin; and proanthocyanidin.

The provided composition may comprise at least curcumin; epigallocatechin-3-gallate; and genistein. The provided composition may comprise at least curcumin; epigallocatechin-3-gallate; and hydroxytyrosol. The provided composition may comprise at least curcumin; epigallocatechin-3-gallate; and Lactobacillus reuteri, Akkermansia mucinophila, and/or Ruminoccocus sp. bacteria. The provided composition may comprise at least curcumin; epigallocatechin-3-gallate; and lycopene. The provided composition may comprise at least curcumin; epigallocatechin-3-gallate; and omega-3 polyunsaturated fatty acid. The provided composition may comprise at least curcumin; epigallocatechin-3-gallate; and quercetin. The provided composition may comprise at least curcumin; epigallocatechin-3-gallate; and proanthocyanidin.

The provided composition may comprise at least curcumin; genistein; and hydroxytyrosol. The provided composition may comprise at least curcumin; genistein; and Lactobacillus reuteri, Akkermansia mucinophila, and/or Ruminoccocus sp. bacteria. The provided composition may comprise at least curcumin; genistein; and lycopene. The provided composition may comprise at least curcumin; genistein; and omega-3 polyunsaturated fatty acid. The provided composition may comprise at least curcumin; genistein; and quercetin. The provided composition may comprise at least curcumin; genistein; and proanthocyanidin.

The provided composition may comprise at least curcumin; hydroxytyrosol; and Lactobacillus reuteri, Akkermansia mucinophila, and/or Ruminoccocus sp. bacteria. The provided composition may comprise at least curcumin; hydroxytyrosol; and lycopene. The provided composition may comprise at least curcumin; hydroxytyrosol; and omega-3 polyunsaturated fatty acid. The provided composition may comprise at least curcumin; hydroxytyrosol; and quercetin. The provided composition may comprise at least curcumin; hydroxytyrosol; and proanthocyanidin.

The provided composition may comprise at least curcumin; Lactobacillus reuteri, Akkermansia mucinophila, and/or Ruminoccocus sp. bacteria; and lycopene. The provided composition may comprise at least curcumin; Lactobacillus reuteri, Akkermansia mucinophila, and/or Ruminoccocus sp. bacteria; and omega-3 polyunsaturated fatty acid. The provided composition may comprise at least curcumin; Lactobacillus reuteri, Akkermansia mucinophila, and/or Ruminoccocus sp. bacteria; and quercetin. The provided composition may comprise at least curcumin; Lactobacillus reuteri, Akkermansia mucinophila, and/or Ruminoccocus sp. bacteria; and proanthocyanidin.

The provided composition may comprise at least curcumin; lycopene; and omega-3 polyunsaturated fatty acid. The provided composition may comprise at least curcumin; lycopene; and quercetin. The provided composition may comprise at least curcumin; lycopene; and proanthocyanidin. The provided composition may comprise at least curcumin; omega-3 polyunsaturated fatty acid; and quercetin. The provided composition may comprise at least curcumin; omega-3 polyunsaturated fatty acid; and proanthocyanidin. The provided composition may comprise at least curcumin; quercetin; and proanthocyanidin.

The provided composition may comprise at least epigallocatechin-3 gallate; genistein; and hydroxytyrosol. The provided composition may comprise at least epigallocatechin-3 gallate; genistein; and Lactobacillus reuteri, Akkermansia mucinophila, and/or Ruminoccocus sp. bacteria. The provided composition may comprise at least epigallocatechin-3 gallate; genistein; and lycopene. The provided composition may comprise at least epigallocatechin-3 gallate; genistein; and omega-3 polyunsaturated fatty acid. The provided composition may comprise at least epigallocatechin-3 gallate; genistein; and quercetin. The provided composition may comprise at least epigallocatechin-3 gallate; genistein; and proanthocyanidin.

The provided composition may comprise at least epigallocatechin-3 gallate; hydroxytyrosol; and Lactobacillus reuteri, Akkermansia mucinophila, and/or Ruminoccocus sp. bacteria. The provided composition may comprise at least epigallocatechin-3 gallate; hydroxytyrosol; and lycopene. The provided composition may comprise at least epigallocatechin-3 gallate; hydroxytyrosol; and omega-3 polyunsaturated fatty acid. The provided composition may comprise at least epigallocatechin-3 gallate; hydroxytyrosol; and quercetin. The provided composition may comprise at least epigallocatechin-3 gallate; hydroxytyrosol; and proanthocyanidin. The provided composition may comprise at least epigallocatechin-3 gallate; Lactobacillus reuteri, Akkermansia mucinophila, and/or Ruminoccocus sp. bacteria; and lycopene. The provided composition may comprise at least epigallocatechin-3 gallate; Lactobacillus reuteri, Akkermansia mucinophila, and/or Ruminoccocus sp. bacteria; and omega-3 polyunsaturated fatty acid. The provided composition may comprise at least epigallocatechin-3 gallate; Lactobacillus reuteri, Akkermansia mucinophila, and/or Ruminoccocus sp. bacteria; and quercetin. The provided composition may comprise at least epigallocatechin-3 gallate; Lactobacillus reuteri, Akkermansia mucinophila, and/or Ruminoccocus sp. bacteria; and proanthocyanidin.

The provided composition may comprise at least epigallocatechin-3 gallate; lycopene; and omega-3 polyunsaturated fatty acid. The provided composition may comprise at least epigallocatechin-3 gallate; lycopene; and quercetin. The provided composition may comprise at least epigallocatechin-3 gallate; lycopene; and proanthocyanidin. The provided composition may comprise at least epigallocatechin-3 gallate; omega-3 polyunsaturated fatty acid; and quercetin. The provided composition may comprise at least epigallocatechin-3 gallate; omega-3 polyunsaturated fatty acid; and proanthocyanidin. The provided composition may comprise at least epigallocatechin-3 gallate; quercetin; and proanthocyanidin.

The provided composition may comprise at least genistein; hydroxytyrosol; and Lactobacillus reuteri, Akkermansia mucinophila, and/or Ruminoccocus sp. bacteria. The provided composition may comprise at least genistein; hydroxytyrosol; and lycopene. The provided composition may comprise at least genistein; hydroxytyrosol; and omega-3 polyunsaturated fatty acid. The provided composition may comprise at least genistein; hydroxytyrosol; and quercetin. The provided composition may comprise at least genistein; hydroxytyrosol; and proanthocyanidin. The provided composition may comprise at least genistein; Lactobacillus reuteri, Akkermansia mucinophila, and/or Ruminoccocus sp. bacteria; and lycopene. The provided composition may comprise at least genistein; Lactobacillus reuteri, Akkermansia mucinophila, and/or Ruminoccocus sp. bacteria; and omega-3 polyunsaturated fatty acid. The provided composition may comprise at least genistein; Lactobacillus reuteri, Akkermansia mucinophila, and/or Ruminoccocus sp. bacteria; and quercetin. The provided composition may comprise at least genistein; Lactobacillus reuteri, Akkermansia mucinophila, and/or Ruminoccocus sp. bacteria; and proanthocyanidin.

The provided composition may comprise at least genistein; lycopene; and omega-3 polyunsaturated fatty acid. The provided composition may comprise at least genistein; lycopene; and quercetin. The provided composition may comprise at least genistein; lycopene; and proanthocyanidin. The provided composition may comprise at least genistein; omega-3 polyunsaturated fatty acid; and quercetin. The provided composition may comprise at least genistein; omega-3 polyunsaturated fatty acid; and proanthocyanidin. The provided composition may comprise at least genistein; quercetin; and proanthocyanidin.

The provided composition may comprise at least hydroxytyrosol; Lactobacillus reuteri, Akkermansia mucinophila, and/or Ruminoccocus sp. bacteria; and lycopene. The provided composition may comprise at least hydroxytyrosol; Lactobacillus reuteri, Akkermansia mucinophila, and/or Ruminoccocus sp. bacteria; and omega-3 polyunsaturated fatty acid. The provided composition may comprise at least hydroxytyrosol; Lactobacillus reuteri, Akkermansia mucinophila, and/or Ruminoccocus sp. bacteria; and quercetin. The provided composition may comprise at least hydroxytyrosol; Lactobacillus reuteri, Akkermansia mucinophila, and/or Ruminoccocus sp. bacteria; and proanthocyanidin.

The provided composition may comprise at least hydroxytyrosol; lycopene; and omega-3 polyunsaturated fatty acid. The provided composition may comprise at least hydroxytyrosol; lycopene; and quercetin. The provided composition may comprise at least hydroxytyrosol; lycopene; and proanthocyanidin. The provided composition may comprise at least hydroxytyrosol; omega-3 polyunsaturated fatty acid; and quercetin. The provided composition may comprise at least hydroxytyrosol; omega-3 polyunsaturated fatty acid; and proanthocyanidin. The provided composition may comprise at least hydroxytyrosol; quercetin; and proanthocyanidin.

The provided composition may comprise at least Lactobacillus reuteri, Akkermansia mucinophila, and/or Ruminoccocus sp. bacteria; lycopene; and omega-3 polyunsaturated fatty acid. The provided composition may comprise at least Lactobacillus reuteri, Akkermansia mucinophila, and/or Ruminoccocus sp. bacteria; lycopene; and quercetin. The provided composition may comprise at least Lactobacillus reuteri, Akkermansia mucinophila, and/or Ruminoccocus sp. bacteria; lycopene; and proanthocyanidin. The provided composition may comprise at least Lactobacillus reuteri, Akkermansia mucinophila, and/or Ruminoccocus sp. bacteria; omega-3 polyunsaturated fatty acid; and quercetin. The provided composition may comprise at least Lactobacillus reuteri, Akkermansia mucinophila, and/or Ruminoccocus sp. bacteria; omega-3 polyunsaturated fatty acid; and proanthocyanidin. The provided composition may comprise at least Lactobacillus reuteri, Akkermansia mucinophila, and/or Ruminoccocus sp. bacteria; quercetin; and proanthocyanidin. The provided composition may comprise at least lycopene; omega-3 polyunsaturated fatty acid; and quercetin. The provided composition may comprise at least lycopene; omega-3 polyunsaturated fatty acid; and proanthocyanidin. The provided composition may comprise at least omega-3 polyunsaturated fatty acid; quercetin and proanthocyanidin.

In any aspect or embodiment, the provided composition may further comprise spices (e.g., cinnamon spice, nutmeg, cumin, clove, turmeric, anise, ginger, cardamom, chili, black pepper, Szechuan pepper, paprika, mustard, etc.). In any aspect or embodiment, the provided composition may further comprise a food product, beverage product, or both. In any aspect or embodiment, the composition may further comprise flavorings, spices, an oil pack, a meal replacement, a functional beverage, a functional food, a medical food, condiments, prescription dietary food, pet food, or any combination thereof. In any aspect or embodiment, the provided composition may comprise a dietary supplement. In any aspect or embodiment, the composition and method of the present invention may comprise dietary sustenance, foods, and/or supplements for human and/or animal space travel, space station settlement, and extraterrestrial settlement.

In any aspect or embodiment, the provided composition may be selected for its odor profile, flavor profile, or both. In any aspect or embodiment, the provided composition may be selected for its solubility. In any aspect or embodiment, the provided composition may be selected for its stability. In any aspect or embodiment, the provided composition may be selected for its potency. In any aspect or embodiment, the provided composition may be selected for its bioavailability.

In any aspect or embodiment, any lycopene contained in the provided composition may comprise all-trans-lycopene, or a mixture of trans- and cis-lycopenes. In any aspect or embodiment, the omega-3 polyunsaturated fatty acids may be derived from marine organisms, including, e.g., fish. In any aspect or embodiment, the Lactobacillus reuteri may be the strain L. reuteri ATCC PTA 6475.

In any aspect or embodiment, the provided composition may further comprise an acceptable carrier, excipient, buffer, or any combination thereof.

In any aspect or embodiment, the provided composition may be delivered in the form of a pill, capsule, cream, salve, syrup, dermal patch, suppository, intravenous drip, aqueous solution, non-aqueous solution, or any combination thereof. In any aspect or embodiment, the provided composition may be in aqueous solution. In any aspect or embodiment, the provided composition may be prepackaged in a container, pack, or syringe. In any aspect or embodiment, the provided composition may further comprise instructions for administration. In any aspect or embodiment, the provided composition may be delivered to a mammalian subject by ingestion, intravenous injection, peritoneal injection, muscular injection, nasally, orally, rectally, or any combination thereof. In any aspect or embodiment, the components of the provided composition are selected in pharmaceutically effective amounts and/or concentrations.

In still another aspect, the present invention comprises a method of activating physiological defenses in a mammalian subject, comprising the steps of (1) selecting and combining three or more of any of anthocyanin; β-(1-3,1-6)-D-glucan; chlorogenic acid; curcumin; epigallocatechin-3-gallate; genistein; hydroxytyrosol; Lactobacillus reuteri, Akkermansia mucinophila, and/or Ruminoccocus sp. bacteria; lycopene; omega-3 polyunsaturated fatty acid; quercetin; and proanthocyanidin; and (2) administering the combination of three or more selections to a mammalian subject. In any embodiment, step (1) may comprise selecting at least four, at least five, at least six, at least seven, at least eight, at least nine, at least ten, at least eleven, or twelve of the above-named twelve entities.

More specifically, the selected composition may comprise at least anthocyanin; β-(1-3,1-6)-D-glucan; and chlorogenic acid. The selected composition may comprise at least anthocyanin; β-(1-3,1-6)-D-glucan; and curcumin. The selected composition may comprise at least anthocyanin; β-(1-3,1-6)-D-glucan; and epigallocatechin-3-gallate. The selected composition may comprise at least anthocyanin; β-(1-3,1-6)-D-glucan; and genistein. The selected composition may comprise at least anthocyanin; β-(1-3,1-6)-D-glucan; and hydroxytyrosol. The selected composition may comprise at least anthocyanin; β-(1-3,1-6)-D-glucan; and Lactobacillus reuteri, Akkermansia mucinophila, and/or Ruminoccocus sp. bacteria. The selected composition may comprise at least anthocyanin; β-(1-3,1-6)-D-glucan; and lycopene. The selected composition may comprise at least anthocyanin; β-(1-3,1-6)-D-glucan; and omega-3 polyunsaturated fatty acid. The selected composition may comprise at least anthocyanin; β-(1-3,1-6)-D-glucan; and quercetin. The selected composition may comprise at least anthocyanin; β-(1-3,1-6)-D-glucan; and proanthocyanidin.

The selected composition may comprise at least anthocyanin; chlorogenic acid; and curcumin. The selected composition may comprise at least anthocyanin; chlorogenic acid; and epigallocatechin-3-gallate. The selected composition may comprise at least anthocyanin; chlorogenic acid; and genistein. The selected composition may comprise at least anthocyanin; chlorogenic acid; and hydroxytyrosol. The selected composition may comprise at least anthocyanin; chlorogenic acid; and Lactobacillus reuteri, Akkermansia mucinophila, and/or Ruminoccocus sp. bacteria. The selected composition may comprise at least anthocyanin; chlorogenic acid; and lycopene. The selected composition may comprise at least anthocyanin; chlorogenic acid; and omega-3 polyunsaturated fatty acid. The selected composition may comprise at least anthocyanin; chlorogenic acid; and quercetin. The selected composition may comprise at least anthocyanin; chlorogenic acid; and proanthocyanidin.

The selected composition may comprise at least anthocyanin; curcumin; and epigallocatechin-3-gallate. The selected composition may comprise at least anthocyanin; curcumin; and genistein. The selected composition may comprise at least anthocyanin; curcumin; and hydroxytyrosol. The selected composition may comprise at least anthocyanin; curcumin; and Lactobacillus reuteri, Akkermansia mucinophila, and/or Ruminoccocus sp. bacteria. The selected composition may comprise at least anthocyanin; curcumin; and lycopene. The selected composition may comprise at least anthocyanin; curcumin; and omega-3 polyunsaturated fatty acid. The selected composition may comprise at least anthocyanin; curcumin; and quercetin. The selected composition may comprise at least anthocyanin; curcumin; and proanthocyanidin.

The selected composition may comprise at least anthocyanin; epigallocatechin-3-gallate; and genistein. The selected composition may comprise at least anthocyanin; epigallocatechin-3-gallate; and hydroxytyrosol. The selected composition may comprise at least anthocyanin; epigallocatechin-3-gallate; and Lactobacillus reuteri, Akkermansia mucinophila, and/or Ruminoccocus sp. bacteria. The selected composition may comprise at least anthocyanin; epigallocatechin-3-gallate; and lycopene. The selected composition may comprise at least anthocyanin; epigallocatechin-3-gallate; and omega-3 polyunsaturated fatty acid. The selected composition may comprise at least anthocyanin; epigallocatechin-3-gallate; and quercetin. The selected composition may comprise at least anthocyanin; epigallocatechin-3-gallate; and proanthocyanidin.

The selected composition may comprise at least anthocyanin; genistein; and hydroxytyrosol. The selected composition may comprise at least anthocyanin; genistein; and Lactobacillus reuteri, Akkermansia mucinophila, and/or Ruminoccocus sp. bacteria. The selected composition may comprise at least anthocyanin; genistein; and lycopene. The selected composition may comprise at least anthocyanin; genistein; and omega-3 polyunsaturated fatty acid. The selected composition may comprise at least anthocyanin; genistein; and quercetin. The selected composition may comprise at least anthocyanin; genistein; and proanthocyanidin.

The selected composition may comprise at least anthocyanin; hydroxytyrosol; and Lactobacillus reuteri, Akkermansia mucinophila, and/or Ruminoccocus sp. bacteria. The selected composition may comprise at least anthocyanin; hydroxytyrosol; and lycopene. The selected composition may comprise at least anthocyanin; hydroxytyrosol; and omega-3 polyunsaturated fatty acid. The selected composition may comprise at least anthocyanin; hydroxytyrosol; and quercetin. The selected composition may comprise at least anthocyanin; hydroxytyrosol; and proanthocyanidin.

The selected composition may comprise at least anthocyanin; Lactobacillus reuteri, Akkermansia mucinophila, and/or Ruminoccocus sp. bacteria; and lycopene. The selected composition may comprise at least anthocyanin; Lactobacillus reuteri, Akkermansia mucinophila, and/or Ruminoccocus sp. bacteria; and omega-3 polyunsaturated fatty acid. The selected composition may comprise at least anthocyanin; Lactobacillus reuteri, Akkermansia mucinophila, and/or Ruminoccocus sp. bacteria; and quercetin. The selected composition may comprise at least anthocyanin; Lactobacillus reuteri, Akkermansia mucinophila, and/or Ruminoccocus sp. bacteria; and proanthocyanidin.

The selected composition may comprise at least anthocyanin; lycopene; and omega-3 polyunsaturated fatty acid. The selected composition may comprise at least anthocyanin; lycopene; and quercetin. The selected composition may comprise at least anthocyanin; lycopene; and proanthocyanidin. The selected composition may comprise at least anthocyanin; omega-3 polyunsaturated fatty acid; and quercetin. The selected composition may comprise at least anthocyanin; omega-3 polyunsaturated fatty acid; and proanthocyanidin. The selected composition may comprise at least anthocyanin; quercetin; and proanthocyanidin.

The selected composition may comprise at least β-(1-3,1-6)-D-glucan; chlorogenic acid; and curcumin. The selected composition may comprise at least β-(1-3,1-6)-D-glucan; chlorogenic acid; and epigallocatechin-3-gallate. The selected composition may comprise at least β-(1-3,1-6)-D-glucan; chlorogenic acid; and genistein. The selected composition may comprise at least β-(1-3,1-6)-D-glucan; chlorogenic acid; and hydroxytyrosol. The selected composition may comprise at least β-(1-3,1-6)-D-glucan; chlorogenic acid; and Lactobacillus reuteri, Akkermansia mucinophila, and/or Ruminoccocus sp. bacteria. The selected composition may comprise at least β-(1-3,1-6)-D-glucan; chlorogenic acid; and lycopene. The selected composition may comprise at least β-(1-3,1-6)-D-glucan; chlorogenic acid; and omega-3 polyunsaturated fatty acid. The selected composition may comprise at least β-(1-3,1-6)-D-glucan; chlorogenic acid; and quercetin. The selected composition may comprise at least β-(1-3,1-6)-D-glucan; chlorogenic acid; and proanthocyanidin.

The selected composition may comprise at least β-(1-3,1-6)-D-glucan; curcumin; and epigallocatechin-3-gallate. The selected composition may comprise at least β-(1-3,1-6)-D-glucan; curcumin; and genistein. The selected composition may comprise at least β-(1-3,1-6)-D-glucan; curcumin; and hydroxytyrosol. The selected composition may comprise at least β-(1-3,1-6)-D-glucan; curcumin; and Lactobacillus reuteri, Akkermansia mucinophila, and/or Ruminoccocus sp. bacteria. The selected composition may comprise at least β-(1-3,1-6)-D-glucan; curcumin; and lycopene. The selected composition may comprise at least J-(1-3,1-6)-D-glucan; curcumin; and omega-3 polyunsaturated fatty acid. The selected composition may comprise at least β-(1-3,1-6)-D-glucan; curcumin; and quercetin. The selected composition may comprise at least β-(1-3,1-6)-D-glucan; curcumin; and proanthocyanidin.

The selected composition may comprise at least β-(1-3,1-6)-D-glucan; epigallocatechin-3-gallate; and genistein. The selected composition may comprise at least β-(1-3,1-6)-D-glucan; epigallocatechin-3-gallate; and hydroxytyrosol. The selected composition may comprise at least β-(1-3,1-6)-D-glucan; epigallocatechin-3-gallate; and Lactobacillus reuteri, Akkermansia mucinophila, and/or Ruminoccocus sp. bacteria. The selected composition may comprise at least β-(1-3,1-6)-D-glucan; epigallocatechin-3-gallate; and lycopene. The selected composition may comprise at least β-(1-3,1-6)-D-glucan; epigallocatechin-3-gallate; and omega-3 polyunsaturated fatty acid. The selected composition may comprise at least β-(1-3,1-6)-D-glucan; epigallocatechin-3-gallate; and quercetin. The selected composition may comprise at least β-(1-3,1-6)-D-glucan; epigallocatechin-3-gallate; and proanthocyanidin.

The selected composition may comprise at least β-(1-3,1-6)-D-glucan; genistein; and hydroxytyrosol. The selected composition may comprise at least β-(1-3,1-6)-D-glucan; genistein; and Lactobacillus reuteri, Akkermansia mucinophila, and/or Ruminoccocus sp. bacteria. The selected composition may comprise at least β-(1-3,1-6)-D-glucan; genistein; and lycopene. The selected composition may comprise at least β-(1-3,1-6)-D-glucan; genistein; and omega-3 polyunsaturated fatty acid. The selected composition may comprise at least β-(1-3,1-6)-D-glucan; genistein; and quercetin. The selected composition may comprise at least β-(1-3,1-6)-D-glucan; genistein; and proanthocyanidin. The selected composition may comprise at least β-(1-3,1-6)-D-glucan; hydroxytyrosol; and Lactobacillus reuteri, Akkermansia mucinophila, and/or Ruminoccocus sp. bacteria. The selected composition may comprise at least β-(1-3,1-6)-D-glucan; hydroxytyrosol; and lycopene. The selected composition may comprise at least β-(1-3,1-6)-D-glucan; hydroxytyrosol; and omega-3 polyunsaturated fatty acid. The selected composition may comprise at least β-(1-3,1-6)-D-glucan; hydroxytyrosol; and quercetin. The selected composition may comprise at least β-(1-3,1-6)-D-glucan; hydroxytyrosol; and proanthocyanidin.

The selected composition may comprise at least β-(1-3,1-6)-D-glucan; Lactobacillus reuteri, Akkermansia mucinophila, and/or Ruminoccocus sp. bacteria; and lycopene. The selected composition may comprise at least β-(1-3,1-6)-D-glucan; Lactobacillus reuteri, Akkermansia mucinophila, and/or Ruminoccocus sp. bacteria; and omega-3 polyunsaturated fatty acid. The selected composition may comprise at least β-(1-3,1-6)-D-glucan; Lactobacillus reuteri, Akkermansia mucinophila, and/or Ruminoccocus sp. bacteria; and quercetin. The selected composition may comprise at least β-(1-3,1-6)-D-glucan; Lactobacillus reuteri, Akkermansia mucinophila, and/or Ruminoccocus sp. bacteria; and proanthocyanidin.

The selected composition may comprise at least β-(1-3,1-6)-D-glucan; lycopene; and omega-3 polyunsaturated fatty acid. The selected composition may comprise at least β-(1-3,1-6)-D-glucan; lycopene; and quercetin. The selected composition may comprise at least β-(1-3,1-6)-D-glucan; lycopene; and proanthocyanidin. The selected composition may comprise at least β-(1-3,1-6)-D-glucan; omega-3 polyunsaturated fatty acid; and quercetin. The selected composition may comprise at least β-(1-3,1-6)-D-glucan; omega-3 polyunsaturated fatty acid; and proanthocyanidin.

The selected composition may comprise at least β-(1-3,1-6)-D-glucan; quercetin; and proanthocyanidin. The selected composition may comprise at least chlorogenic acid; curcumin; and epigallocatechin-3-gallate. The selected composition may comprise at least chlorogenic acid; curcumin; and genistein. The selected composition may comprise at least chlorogenic acid; curcumin; and hydroxytyrosol. The selected composition may comprise at least chlorogenic acid; curcumin; and Lactobacillus reuteri, Akkermansia mucinophila, and/or Ruminoccocus sp. bacteria. The selected composition may comprise at least chlorogenic acid; curcumin; and lycopene. The selected composition may comprise at least chlorogenic acid; curcumin; and omega-3 polyunsaturated fatty acid. The selected composition may comprise at least chlorogenic acid; curcumin; and quercetin. The selected composition may comprise at least chlorogenic acid; curcumin; and proanthocyanidin.

The selected composition may comprise at least chlorogenic acid; epigallocatechin-3-gallate; and genistein. The selected composition may comprise at least chlorogenic acid; epigallocatechin-3-gallate; and hydroxytyrosol. The selected composition may comprise at least chlorogenic acid; epigallocatechin-3-gallate; and Lactobacillus reuteri, Akkermansia mucinophila, and/or Ruminoccocus sp. bacteria. The selected composition may comprise at least chlorogenic acid; epigallocatechin-3-gallate; and lycopene. The selected composition may comprise at least chlorogenic acid; epigallocatechin-3-gallate; and omega-3 polyunsaturated fatty acid. The selected composition may comprise at least chlorogenic acid; epigallocatechin-3-gallate; and quercetin. The selected composition may comprise at least chlorogenic acid; epigallocatechin-3-gallate; and proanthocyanidin.

The selected composition may comprise at least chlorogenic acid; genistein; and hydroxytyrosol. The selected composition may comprise at least chlorogenic acid; genistein; and Lactobacillus reuteri, Akkermansia mucinophila, and/or Ruminoccocus sp. bacteria. The selected composition may comprise at least chlorogenic acid; genistein; and lycopene. The selected composition may comprise at least chlorogenic acid; genistein; and omega-3 polyunsaturated fatty acid. The selected composition may comprise at least chlorogenic acid; genistein; and quercetin. The selected composition may comprise at least chlorogenic acid; genistein; and proanthocyanidin. The selected composition may comprise at least chlorogenic acid; hydroxytyrosol; and Lactobacillus reuteri, Akkermansia mucinophila, and/or Ruminoccocus sp. bacteria. The selected composition may comprise at least chlorogenic acid; hydroxytyrosol; and lycopene. The selected composition may comprise at least chlorogenic acid; hydroxytyrosol; and omega-3 polyunsaturated fatty acid. The selected composition may comprise at least chlorogenic acid; hydroxytyrosol; and quercetin. The selected composition may comprise at least chlorogenic acid; hydroxytyrosol; and proanthocyanidin.

The selected composition may comprise at least chlorogenic acid; Lactobacillus reuteri, Akkermansia mucinophila, and/or Ruminoccocus sp. bacteria; and lycopene. The selected composition may comprise at least chlorogenic acid; Lactobacillus reuteri, Akkermansia mucinophila, and/or Ruminoccocus sp. bacteria; and omega-3 polyunsaturated fatty acid. The selected composition may comprise at least chlorogenic acid; Lactobacillus reuteri, Akkermansia mucinophila, and/or Ruminoccocus sp. bacteria; and quercetin. The selected composition may comprise at least chlorogenic acid; Lactobacillus reuteri, Akkermansia mucinophila, and/or Ruminoccocus sp. bacteria; and proanthocyanidin.

The selected composition may comprise at least chlorogenic acid; lycopene; and omega-3 polyunsaturated fatty acid. The selected composition may comprise at least chlorogenic acid; lycopene; and quercetin. The selected composition may comprise at least chlorogenic acid; lycopene; and proanthocyanidin. The selected composition may comprise at least chlorogenic acid; omega-3 polyunsaturated fatty acid; and quercetin. The selected composition may comprise at least chlorogenic acid; omega-3 polyunsaturated fatty acid; and proanthocyanidin. The selected composition may comprise at least chlorogenic acid; quercetin; and proanthocyanidin.

The selected composition may comprise at least curcumin; epigallocatechin-3-gallate; and genistein. The selected composition may comprise at least curcumin; epigallocatechin-3-gallate; and hydroxytyrosol. The selected composition may comprise at least curcumin; epigallocatechin-3-gallate; and Lactobacillus reuteri, Akkermansia mucinophila, and/or Ruminoccocus sp. bacteria. The selected composition may comprise at least curcumin; epigallocatechin-3-gallate; and lycopene. The selected composition may comprise at least curcumin; epigallocatechin-3-gallate; and omega-3 polyunsaturated fatty acid. The selected composition may comprise at least curcumin; epigallocatechin-3-gallate; and quercetin. The selected composition may comprise at least curcumin; epigallocatechin-3-gallate; and proanthocyanidin.

The selected composition may comprise at least curcumin; genistein; and hydroxytyrosol. The selected composition may comprise at least curcumin; genistein; and Lactobacillus reuteri, Akkermansia mucinophila, and/or Ruminoccocus sp. bacteria. The selected composition may comprise at least curcumin; genistein; and lycopene. The selected composition may comprise at least curcumin; genistein; and omega-3 polyunsaturated fatty acid. The selected composition may comprise at least curcumin; genistein; and quercetin. The selected composition may comprise at least curcumin; genistein; and proanthocyanidin.

The selected composition may comprise at least curcumin; hydroxytyrosol; and Lactobacillus reuteri, Akkermansia mucinophila, and/or Ruminoccocus sp. bacteria. The selected composition may comprise at least curcumin; hydroxytyrosol; and lycopene. The selected composition may comprise at least curcumin; hydroxytyrosol; and omega-3 polyunsaturated fatty acid. The selected composition may comprise at least curcumin; hydroxytyrosol; and quercetin. The selected composition may comprise at least curcumin; hydroxytyrosol; and proanthocyanidin.

The selected composition may comprise at least curcumin; Lactobacillus reuteri, Akkermansia mucinophila, and/or Ruminoccocus sp. bacteria; and lycopene. The selected composition may comprise at least curcumin; Lactobacillus reuteri, Akkermansia mucinophila, and/or Ruminoccocus sp. bacteria; and omega-3 polyunsaturated fatty acid. The selected composition may comprise at least curcumin; Lactobacillus reuteri, Akkermansia mucinophila, and/or Ruminoccocus sp. bacteria; and quercetin. The selected composition may comprise at least curcumin; Lactobacillus reuteri, Akkermansia mucinophila, and/or Ruminoccocus sp. bacteria; and proanthocyanidin.

The selected composition may comprise at least curcumin; lycopene; and omega-3 polyunsaturated fatty acid. The selected composition may comprise at least curcumin; lycopene; and quercetin. The selected composition may comprise at least curcumin; lycopene; and proanthocyanidin. The selected composition may comprise at least curcumin; omega-3 polyunsaturated fatty acid; and quercetin. The selected composition may comprise at least curcumin; omega-3 polyunsaturated fatty acid; and proanthocyanidin. The selected composition may comprise at least curcumin; quercetin; and proanthocyanidin.

The selected composition may comprise at least epigallocatechin-3 gallate; genistein; and hydroxytyrosol. The selected composition may comprise at least epigallocatechin-3 gallate; genistein; and Lactobacillus reuteri, Akkermansia mucinophila, and/or Ruminoccocus sp. bacteria. The selected composition may comprise at least epigallocatechin-3 gallate; genistein; and lycopene. The selected composition may comprise at least epigallocatechin-3 gallate; genistein; and omega-3 polyunsaturated fatty acid. The selected composition may comprise at least epigallocatechin-3 gallate; genistein; and quercetin. The selected composition may comprise at least epigallocatechin-3 gallate; genistein; and proanthocyanidin.

The selected composition may comprise at least epigallocatechin-3 gallate; hydroxytyrosol; and Lactobacillus reuteri, Akkermansia mucinophila, and/or Ruminoccocus sp. bacteria. The selected composition may comprise at least epigallocatechin-3 gallate; hydroxytyrosol; and lycopene. The selected composition may comprise at least epigallocatechin-3 gallate; hydroxytyrosol; and omega-3 polyunsaturated fatty acid. The selected composition may comprise at least epigallocatechin-3 gallate; hydroxytyrosol; and quercetin. The selected composition may comprise at least epigallocatechin-3 gallate; hydroxytyrosol; and proanthocyanidin. The selected composition may comprise at least epigallocatechin-3 gallate; Lactobacillus reuteri, Akkermansia mucinophila, and/or Ruminoccocus sp. bacteria; and lycopene. The selected composition may comprise at least epigallocatechin-3 gallate; Lactobacillus reuteri, Akkermansia mucinophila, and/or Ruminoccocus sp. bacteria; and omega-3 polyunsaturated fatty acid. The selected composition may comprise at least epigallocatechin-3 gallate; Lactobacillus reuteri, Akkermansia mucinophila, and/or Ruminoccocus sp. bacteria; and quercetin. The selected composition may comprise at least epigallocatechin-3 gallate; Lactobacillus reuteri, Akkermansia mucinophila, and/or Ruminoccocus sp. bacteria; and proanthocyanidin.

The selected composition may comprise at least epigallocatechin-3 gallate; lycopene; and omega-3 polyunsaturated fatty acid. The selected composition may comprise at least epigallocatechin-3 gallate; lycopene; and quercetin. The selected composition may comprise at least epigallocatechin-3 gallate; lycopene; and proanthocyanidin. The selected composition may comprise at least epigallocatechin-3 gallate; omega-3 polyunsaturated fatty acid; and quercetin. The selected composition may comprise at least epigallocatechin-3 gallate; omega-3 polyunsaturated fatty acid; and proanthocyanidin. The selected composition may comprise at least epigallocatechin-3 gallate; quercetin; and proanthocyanidin.

The selected composition may comprise at least genistein; hydroxytyrosol; and Lactobacillus reuteri, Akkermansia mucinophila, and/or Ruminoccocus sp. bacteria. The selected composition may comprise at least genistein; hydroxytyrosol; and lycopene. The selected composition may comprise at least genistein; hydroxytyrosol; and omega-3 polyunsaturated fatty acid. The selected composition may comprise at least genistein; hydroxytyrosol; and quercetin. The selected composition may comprise at least genistein; hydroxytyrosol; and proanthocyanidin. The selected composition may comprise at least genistein; Lactobacillus reuteri, Akkermansia mucinophila, and/or Ruminoccocus sp. bacteria; and lycopene. The selected composition may comprise at least genistein; Lactobacillus reuteri, Akkermansia mucinophila, and/or Ruminoccocus sp. bacteria; and omega-3 polyunsaturated fatty acid. The selected composition may comprise at least genistein; Lactobacillus reuteri, Akkermansia mucinophila, and/or Ruminoccocus sp. bacteria; and quercetin. The selected composition may comprise at least genistein; Lactobacillus reuteri, Akkermansia mucinophila, and/or Ruminoccocus sp. bacteria; and proanthocyanidin.

The selected composition may comprise at least genistein; lycopene; and omega-3 polyunsaturated fatty acid. The selected composition may comprise at least genistein; lycopene; and quercetin. The selected composition may comprise at least genistein; lycopene; and proanthocyanidin. The selected composition may comprise at least genistein; omega-3 polyunsaturated fatty acid; and quercetin. The selected composition may comprise at least genistein; omega-3 polyunsaturated fatty acid; and proanthocyanidin. The selected composition may comprise at least genistein; quercetin; and proanthocyanidin.

The selected composition may comprise at least hydroxytyrosol; Lactobacillus reuteri, Akkermansia mucinophila, and/or Ruminoccocus sp. bacteria; and lycopene. The selected composition may comprise at least hydroxytyrosol; Lactobacillus reuteri, Akkermansia mucinophila, and/or Ruminoccocus sp. bacteria; and omega-3 polyunsaturated fatty acid. The selected composition may comprise at least hydroxytyrosol; Lactobacillus reuteri, Akkermansia mucinophila, and/or Ruminoccocus sp. bacteria; and quercetin. The selected composition may comprise at least hydroxytyrosol; Lactobacillus reuteri, Akkermansia mucinophila, and/or Ruminoccocus sp. bacteria; and proanthocyanidin.

The selected composition may comprise at least hydroxytyrosol; lycopene; and omega-3 polyunsaturated fatty acid. The selected composition may comprise at least hydroxytyrosol; lycopene; and quercetin. The selected composition may comprise at least hydroxytyrosol; lycopene; and proanthocyanidin. The selected composition may comprise at least hydroxytyrosol; omega-3 polyunsaturated fatty acid; and quercetin. The selected composition may comprise at least hydroxytyrosol; omega-3 polyunsaturated fatty acid; and proanthocyanidin. The selected composition may comprise at least hydroxytyrosol; quercetin; and proanthocyanidin.

The selected composition may comprise at least Lactobacillus reuteri, Akkermansia mucinophila, and/or Ruminoccocus sp. bacteria; lycopene; and omega-3 polyunsaturated fatty acid. The selected composition may comprise at least Lactobacillus reuteri, Akkermansia mucinophila, and/or Ruminoccocus sp. bacteria; lycopene; and quercetin. The selected composition may comprise at least Lactobacillus reuteri, Akkermansia mucinophila, and/or Ruminoccocus sp. bacteria; lycopene; and proanthocyanidin. The selected composition may comprise at least Lactobacillus reuteri, Akkermansia mucinophila, and/or Ruminoccocus sp. bacteria; omega-3 polyunsaturated fatty acid; and quercetin. The selected composition may comprise at least Lactobacillus reuteri, Akkermansia mucinophila, and/or Ruminoccocus sp. bacteria; omega-3 polyunsaturated fatty acid; and proanthocyanidin. The selected composition may comprise at least Lactobacillus reuteri, Akkermansia mucinophila, and/or Ruminoccocus sp. bacteria; quercetin; and proanthocyanidin. The selected composition may comprise at least lycopene; omega-3 polyunsaturated fatty acid; and quercetin. The selected composition may comprise at least lycopene; omega-3 polyunsaturated fatty acid; and proanthocyanidin. The selected composition may comprise at least omega-3 polyunsaturated fatty acid; quercetin and proanthocyanidin.

In any aspect or embodiment, the selected composition may further comprise spices (e.g., cinnamon spice, nutmeg, cumin, clove, turmeric, anise, ginger, cardamom, chili, black pepper, Szechuan pepper, paprika, mustard, etc.). In any aspect or embodiment, the selected composition may further comprise a food product, beverage product, or both. In any aspect or embodiment, the selected composition may further comprise flavorings, spices, an oil pack, a meal replacement, a functional beverage, a functional food, a medical food, condiments, prescription dietary food, pet food, or any combination thereof. In any aspect or embodiment, the composition and method of the present invention may comprise dietary sustenance, foods, and/or supplements for human and/or animal space travel, space station settlement, and extraterrestrial settlement.

In any aspect or embodiment, the selected composition may be selected for its odor profile, flavor profile, or both. In any aspect or embodiment, the selected composition may be selected for its solubility. In any aspect or embodiment, the selected composition may be selected for its stability. In any aspect or embodiment, the selected composition may be selected for its potency. In any aspect or embodiment, the selected composition may be selected for its bioavailability.

In any aspect or embodiment, the lycopene may comprise all-trans-lycopene, or a mixture of trans- and cis-lycopenes. In any aspect or embodiment, the omega-3 polyunsaturated fatty acids may be derived from marine organisms, including, e.g., fish. In any aspect or embodiment, the Lactobacillus reuteri may be the strain L. reuteri ATCC PTA 6475.

In any aspect or embodiment, the selected composition may further comprise an acceptable carrier, excipient, buffer, or any combination thereof.

In any aspect or embodiment, the selected composition may be delivered in the form of a pill, capsule, cream, salve, syrup, dermal patch, dissolvable strip (e.g., to be dissolved on a subject's tongue), suppository, intravenous drip, aqueous solution, non-aqueous solution, powder (e.g., to be dissolved in aqueous solvent), or any combination thereof. In any aspect or embodiment, the selected composition may be in aqueous solution. In any aspect or embodiment, the selected composition may be prepackaged in a container, pack, or syringe. In any aspect or embodiment, the selected composition may further comprise instructions for administration. In any aspect or embodiment, the selected composition may be administered to a mammalian subject by ingestion, intravenous injection, peritoneal injection, muscular injection, nasally, orally, rectally, or any combination thereof. In any aspect or embodiment, the components of the composition are selected in pharmaceutically effective amounts and/or concentrations.

In any aspect or embodiment, the mammalian subject may be a human subject, such as, e.g., a human patient.

In some embodiments, the pharmaceutical composition provided herein comprise a therapeutically effective amount of the composition of the present invention or pharmaceutically acceptable salt thereof in admixture with a pharmaceutically acceptable carrier, diluent and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for at least six months at 4° C.

In some embodiments, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. The pH of the buffer may be in the range of 6.5 to 7.75, preferably 7 to 7.5, and most preferably 7.2 to 7.4.

The invention described herein further comprises a food product, wherein the food product may be a human food product, a dietary supplement formulated for humans and/or non-human mammals, or a pet food. The food product may comprise at least three of the twelve functional entities selected from (1) anthocyanins; (2) β-(1-3,1-6)-D-glucan; (3) chlorogenic acid; (4) curcumin; (5) epigallocatechin-3-gallate (EGCG); (6) genistein; (7) hydroxytyrosol; (8) lycopene (including both cis- and trans-isomers); (9) Lactobacillus reuteri, Akkermansia mucinophila, and/or Ruminoccocus sp. bacteria; (10) omega-3 polyunsaturated fatty acid (PUFA); (11) quercetin; and (12) proanthocyanidins. The food product may be a functional food. The food product may comprise a soup, which may be dehydrated or hydrated, and may comprise broth, or may comprise at least broth and noodles. The twelve functional entities may be incorporated into the soup as extracts, synthetics, or in a whole food selected for its high concentration of at least one of the twelve functional entities (e.g., tomatoes having a high concentration in lycopene).

The dietary supplement may comprise a solid or a liquid. The solid may comprise, e.g., a powder and may be water-soluble or water-soluble or amphiphilic. The solid may be packaged in a pill or capsule. The liquid may be aqueous or non-aqueous, and may be mixed into foods, drinks, or ingested as a standalone.

The pet food may comprise dry food (i.e., kibble) or wet food. The pet food may be formulated for any commonly domesticated mammal, e.g., dog, cat, horse, cow, pig, hamster, gerbil, guinea pig, mouse, rat, ferret, or chinchilla. The pet food may be formulated as standalone pet food, i.e., to be served to the pet regularly in whole servings meet the pet's entire dietary needs; or the pet food may be formulated as a supplement, i.e., to be served together with or as a supplement to the diet the caregiver as otherwise chosen for the pet.

In still another aspect, the invention further comprises a noodle soup kit, wherein the kit comprises dehydrated noodles, a dehydrated soup base, and at least three of functional entities, wherein the functional entities are selected from the group consisting of: anthocyanins; β-(1-3,1-6)-D-glucan; chlorogenic acid; curcumin; epigallocatechin-3-gallate (EGCG); genistein; hydroxytyrosol; lycopene (including both cis- and trans-isomers); Lactobacillus reuteri, Akkermansia mucinophila, and/or Ruminoccocus sp. bacteria; omega-3 polyunsaturated fatty acid (PUFA); quercetin; and proanthocyanidins. In the noodle soup kit, the twelve functional entities may be incorporated into the soup as extracts, synthetics, or in a whole food selected for its high concentration of at least one of the twelve functional entities (e.g., tomatoes having a high concentration in lycopene).

For example, the noodle soup kit may essentially comprise a ramen noodle package containing at least a mass of dehydrated noodles and a soup base, wherein the soup base may comprise only a dehydrated powder, or may comprise a dehydrated powder and an oil. The noodle soup kit may incorporate at least three of the twelve functional entities into the noodles themselves, into the soup base (into the dehydrated powder or, if any, the oil, or both), or the at least three of the twelve functional entities may be provided separately for the consumer to combine with the noodles and soup base while preparing the noodle soup for consumption.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments. These and other changes can be made to the embodiments in light of the above-detailed description.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A composition comprising any combination of at least three of:
anthocyanin;
β-(1-3,1-6)-D-glucan;
chlorogenic acid;
curcumin;
epigallocatechin-3-gallate;
genistein;
hydroxytyrosol;
Lactobacillus reuteri, Akkermansia mucinophila, and/or Ruminoccocus sp. bacteria;
lycopene;
omega-3 polyunsaturated fatty acid;
quercetin; and
proanthocyanidin.

2. The composition of claim 1 wherein the composition further comprises a food or beverage product, or supplement.

3. The composition of claim 1 wherein the composition further comprises any of: flavoring, spice, oil pack, meal replacement, functional beverage, functional food, medical food, condiments, prescription dietary food, pet food, or supplement.

4. The composition of claim 1 wherein the composition further comprises spices.

5. The composition of claim 1 wherein the combination is selected for desirable flavor profile.

6. The composition of claim 1 wherein the combination is selected for desirable solubility.

7. The composition of claim 1 wherein the combination is selected for desirable stability.

8. The composition of claim 1 wherein the combination is selected for desirable potency.

9. The composition of claim 1 wherein the combination is selected for desirable bioavailability.

10. A method of providing an activator of physiological defenses in a mammalian subject comprising the steps of:
selecting and combining three or more from the group consisting of:
anthocyanin; β-(1-3,1-6)-D-glucan; chlorogenic acid; curcumin; epigallocatechin-3-gallate; genistein; hydroxytyrosol; Lactobacillus reuteri, Akkermansia mucinophila, and/or Ruminoccocus sp. bacteria; lycopene; omega-3 polyunsaturated fatty acid; quercetin; and proanthocyanidin.

11. The method of claim 10 wherein the composition further comprises a food or beverage product, or supplement.

12. The method of claim 10 wherein the composition further comprises any of: flavoring, spice, oil pack, meal replacement, functional beverage, functional food, medical food, condiments, prescription dietary food, pet food, or supplement.

13. The method of claim 10 wherein the composition further comprises spices.

14. The method of claim 10 wherein the combination is selected for desirable flavor profile.

15. The method of claim 10 wherein the combination is selected for desirable solubility.

16. The method of claim 10 wherein the combination is selected for desirable stability.

17. The method of claim 10 wherein the combination is selected for desirable potency.

18. The method of claim 10 wherein the combination is selected for desirable bioavailability.

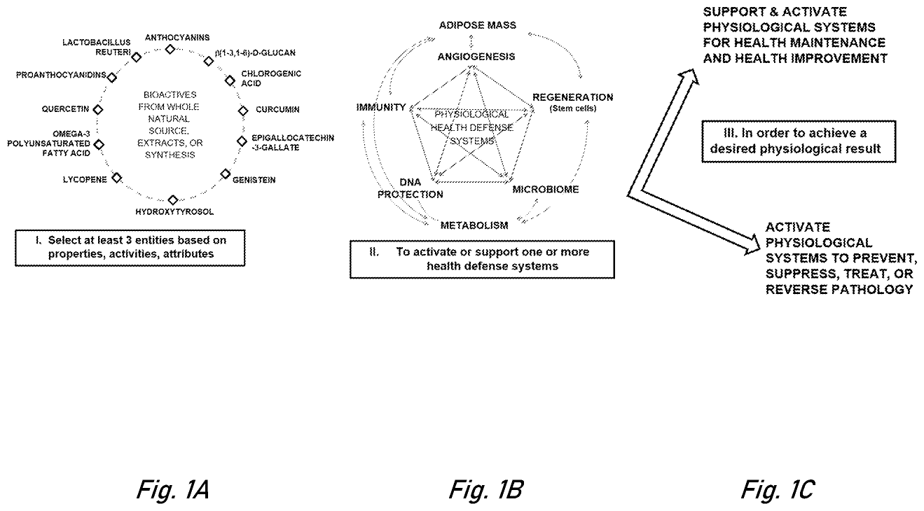

19. The method of claim 10 wherein the combination is selected according to the rubric illustrated in FIGS. 1A, 1B, and 1C.

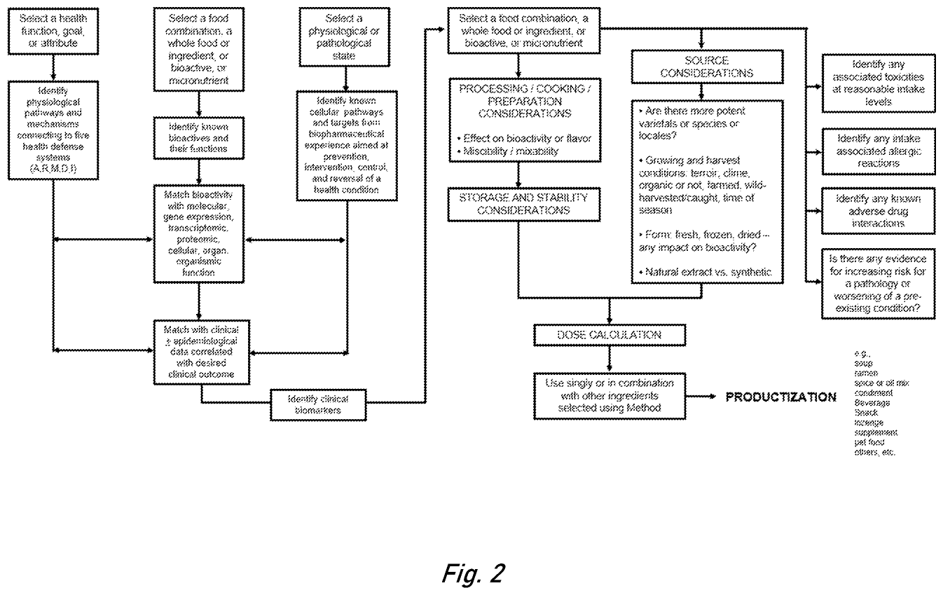

20. The method of claim 10 wherein the combination is selected according to the rubric illustrated in FIG. 2.
